SEQUENCE LISTING (2 sequences)

SEQ ID NO: 1 - RNA, 13 nt, synthetic construct
  source 1..13: mol_type = other RNA
aaatttttaaaat

SEQ ID NO: 2 - RNA, 11 nt, synthetic construct
  source 1..11: mol_type = other RNA
aaatttttaaa

(19) United States
(12) Patent Application Publication
(10) Pub. No.: US 2026/0226153 A1
(43) Pub. Date: Aug. 6, 2026

(54) USE OF INTERFERON-GAMMA INHIBITORS TO REDUCE HEART DAMAGE AFTER MYOCARDIAL INFARCTION

(71) Applicant: Northwestern University, Evanston, IL (US)

(72) Inventors: Samuel E. Weinberg, Evanston, IL (US); Hossein Ardehali, Evanston, IL (US); Amir Mahmoodzadeh, Evanston, IL (US)

(21) Appl. No.: 19/453,569

(22) Filed: Jan. 20, 2026

Related U.S. Application Data
(60) Provisional application No. 63/746,777, filed on Jan. 17, 2025.

(51) Int. Cl. C07K 16/24; A61K 9/00; A61K 31/519; A61K 38/00; A61K 45/06; A61P 9/10

(52) U.S. Cl. CPC C07K 16/249; A61K 9/0019; A61K 31/519; A61K 38/00; A61K 45/06; A61P 9/10

(57) ABSTRACT

The present disclosure provides methods for the treatment of myocardial infarction comprising administering at least one of n interferon gamma (IFNγ) inhibitor and an interleukin-6 (IL-6) inhibitor.

### CROSS-REFERENCE TO RELATED APPLICATIONS

This application claims priority to U.S. Provisional Application No. 63/746,777 filed on Jan. 17, 2025. The contents of which is incorporated by reference in its entirety.

### STATEMENT REGARDING FEDERALLY SPONSORED RESEARCH

This invention was made with government support under grant numbers HL155953 and HL140973 awarded by the National Institutes of Health. The government has certain rights in this invention.

### REFERENCE TO AN ELECTRONIC SEQUENCE LISTING

The contents of the electronic sequence listing (70258102755.xml; Size: 2,687 bytes; and Date of Creation: Jan. 16, 2026) is herein incorporated by reference in its entirety.

### BACKGROUND

Myocardial infarction (MI) is a leading cause of death worldwide. Although major progress has been made in the post-MI care, morbidity and mortality remains high. MI leads to the death of cardiomyocytes (CM) in the region where the blood supply is compromised (referred to as infarct zone or IZ). Additionally, the area around the infarcted region (referred to as border zone or BZ) also has reduced blood flow and plays an important role in cardiac remodeling. The cells within the BZ display a distinct gene expression pattern. The BZ plays a critical role in regulating infarct expansion, fibrosis, repair of the IZ and the region around it, as well as electric instability that may lead to arrhythmias. The BZ may become hypocontractile and the myocardium in the BZ may be more susceptible to injury with additional ischemia and the extension of MI at the same time as the area attempts to undergo the process of remodeling. Additionally, inflammatory cells infiltrate the IZ and BZ to repair the damaged area and help with the remodeling of the BZ. Any disruption in this reparative process can potentially lead to worsened damage; however, there is also opportunity to alter the immune function in the BZ to improve the repair process. Given that CMs within the IZ die after MI, it is challenging to target that region to improve cardiac function after ischemic injury. However, the key to cardiac recovery is to improve remodeling and salvage CMs in the BZ. Accordingly, there is a remaining need in the art for novel targets and methods to improve remodeling in the BZ following MI.

### SUMMARY

The present disclosure provides methods for treating myocardial infarction. In one aspect, the present disclosure provides method of treating myocardial infarction (MI) in a subject in need, the method comprising administering to the subject a therapeutically effective amount of at least one of an interferon-gamma (IFNγ) inhibitor and an interleukin 6 (IL-6) inhibitor. In some embodiments, the method comprises administering the IFNγ inhibitor. In some embodiments, the IFNγ inhibitor reduces IFNγ expression. In some embodiments, the IFNγ inhibitor reduces IFNγ activity. In some embodiments, the IFNγ inhibitor comprises at least one of an antibody, a small molecule drug, a peptide, a gene silencing agent, a protein degrader, and a cytokine. In some embodiments, the IFNγ inhibitor comprises an IFNγ antibody. In some embodiments, the IFNγ antibody is Emapalumab. In some embodiments, about 25 ng/kg and about 50 ng/kg of the IFNγ antibody is administered. In some embodiments, about 50 ng/kg of the IFNγ antibody is administered. In some embodiments, the IFNγ antibody is administered subcutaneously or intravenously. In some embodiments, the IFNγ inhibitor comprises a Jak/Stat inhibitor. In some embodiments, the Jak/Stat inhibitor comprises Ruxolitinib. In some embodiments, the IFNγ inhibitor is administered at least once. In some embodiments, the inhibitor is administered between about 1 day and about 3 days following an MI. In some embodiments the method further comprises administering at least one additional therapy for myocardial infarction. In some embodiments, the at least one additional therapy is selected from the group consisting of an anticoagulant, an antianginal, a narcotic, a beta blocker, a statin, an ACE inhibitor, a surgery, a stent, and an angioplasty. In some embodiments, the subject is a human. In some embodiments, the method comprises administering the IL-6 inhibitor. In some embodiments, the subject expresses elevated TTP levels in border zone cardiomyocytes. In some embodiments, the subject express reduced α-KGDH activity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will be better understood and features, aspects, and advantages other than those set forth above will become apparent when consideration is given to the following detailed description thereof. Such detailed description makes reference to the following drawings.

FIGS. 1A-1N. Metabolic analysis of the BZ displays a block at the level of α-KGDH. (A) Schematic workflow for surgical procedure on mice and sample collection. (B) Masson's Trichrome staining of myocardial sections 7 days after coronary artery ligation, exhibiting distinct histopathological characteristics across the RZ, BZ, and IZ. (C) Levels of TCA metabolites upstream of α-KGDH in RZ and BZ (n=4 mice per group, two-tailed unpaired t-test). (D) Levels of certain TCA metabolites downstream of α-KGDH in RZ and BZ (n=4 mice per group, two-tailed unpaired t-test). (E) α-KGDH activity in BZ and RZ from heart tissue 7 days after coronary artery ligation (n=4 mice per group, two-tailed unpaired t-test). (F,G) Summary of the basal (F) and maximal (G) respiration derived from RZ and BZ heart tissues 7 days after coronary artery ligation (n=3 mice per group, two-tailed unpaired t-test). (H, I) NAD/NADH ratio (H) and ATP levels in the BZ and RZ (n=3 mice per group, two-tailed unpaired t-test). (J) Schematic workflow for surgical procedure and in vivo tracing experiments in mice and sample collection. (K) Schematic presentation of [13C6]-glucose carbon labeling through TCA cycle. (L) Fractional enrichment of metabolites upstream of α-KGDH (i.e., citrate, aconitate and α-KG) in the BZ and RZ (n=4 mice per group, two-tailed unpaired t-test). (M) Fractional enrichment of metabolites downstream of α-KGDH (i.e., succinate, fumarate and malate) in the BZ and RZ (n=4 mice per group, two-tailed unpaired t-test). (N) Schematic presentation of the TCA cycle and the blockage at the level of α-KGDH in the BZ of the heart after MI. Green text represents the metabolites that are increased in our metabolomic studies.

FIGS. 2A-2J. The BZ displays reduced LIAS levels and lipoylation of DLST. (A) Schematic presentation of α-KGDH and its activation by lipoylation. The E2 component binds to lipoyl moieties and the protein becomes activated. (B) Relative mRNA expression of the three components of α-KGDH (i.e., Ogdh, Dlst and Dld) in the BZ and RZ (n=4 mice per group, two-tailed unpaired t-test). (C) Western blot of OGDH, DLST, and DLD in the RZ and BZ. GAPDH serves as the loading control. (D) Western blot densitometry analysis of the membranes in Panel C (n=4 mice per group, two-tailed unpaired t-test). (E) Western blot of LIAS and Lipo-DLST in the RZ and BZ. GAPDH serves as the loading control. (F) Western blot densitometry analysis of the membranes in Panel E (n=4 mice per group, two-tailed unpaired t test). (G) Immunofluorescence analysis of LIAS normalized to DAPI staining in the RZ and BZ from human heart samples after ST-elevation myocardial infarction (STEMI) (n=2 patients per group, two-tailed unpaired t test). (H) LIAS fluorescence intensity in the RZ and BZ from panel G (n=2 samples per group, two-tailed unpaired t test). (I) Immunofluorescence analysis of lipoylated proteins normalized to DAPI in the RZ and BZ from human heart samples after STEMI (n=2 patients per group, two-tailed unpaired t test). (J) Fluorescence intensity of lipoylated proteins in the RZ and BZ from panel G (n=2 samples per group, two-tailed unpaired t test).

FIGS. 3A-3T. TTP is increased in the BZ and reduces DLST and LIAS protein levels. (a) Venn diagram showing overlap between downregulated genes in the BZ and target genes of mRNA-binding proteins. (b) Bar graph showing fold change of RNA-binding proteins in the BZ compared to the RZ, with TTP showing the highest upregulation. Data derived from RNA-seq (GSE183168) [3]. (c) Relative mRNA expression of Ttp, in the RZ and BZ WT mice. Mouse 18S rRNA was used as the internal control for normalization (n=6 mice per group, unpaired t test). (d) Western blot analysis of TTP protein levels in the RZ and BZ. GAPDH serves as the loading control. (e) Western blot densitometry analysis of TTP protein levels normalized to GAPDH in the BZ and RZ from panel (d) in FIG. 2 (n=3 mice per group, two-tailed unpaired t-test). (f) Schematic of the Ttp-floxed allele and αMHC-Cre strategy used to generate cs-Ttp^{-/-} mice. (g) Western blot showing the deletion of TTP protein in the hearts of cs-Ttp^{-/-} mice, with GAPDH as the loading control. (H-J) Western blot densitometry analysis of LIAS and Lipo-DLST protein levels in RZ and BZ from cs-Ttp^{-/-} mice (n=4 mice per group, two-tailed unpaired t-test). (K) RNA co-IP showing TTP binding to Lias, Hprt1, and Tnf-α mRNAs in H9c2 cells. IgG was used as a control for the pulldown studies (n=4 biological replicates, two-tailed unpaired t-test). (L) mRNA decay assay showing Lias transcript stability in H9c2 cells overexpressing eGFP, C124R TTP mutant, or WT TTP. Actinomycin D (ActD) was used to inhibit transcription (n=3 biological replicates, two-way ANOVA with Tukey's post hoc test). (M-O) Western blot and densitometry of LIAS and Lipo-DLST in H9c2 cells overexpressing C124R TTP mutant or WT TTP. GAPDH serves as the loading control. (P) α-KGDH activity in the RZ and BZ from cs-Ttp−/− mice (n=4 mice per group, two-tailed unpaired t-test). (Q) Fractional enrichment of pyruvate (M0 and M3 isotopologues) in the RZ and BZ following in vivo 13C6-glucose tracing. (R) Fractional enrichment of lactate (M0 and M3 isotopologues) in the RZ and BZ following in vivo 13C66-glucose tracing. (S) Pyruvate dehydrogenase (PDH) activity measured in the RZ and BZ of WT (S) and cs-Ttp^{-/-} (T) 7 days pot-MI, normalized to tissue fresh weight. Data are presented as mean±SEM; NS, not significant (n=4 mice per group, two-tailed unpaired t-test).

FIGS. 4A-4J. Deletion of Ttp reverses the metabolic changes of the BZ after MI. (A) Schematic workflow for surgical procedure on cs-Ttp−/− mice and sample collection. (B) Levels of pyruvate and TCA metabolites upstream of α-KGDH in RZ and BZ from cs-Ttp−/− mice 7 days after coronary artery ligation (n=4 mice per group, two-tailed unpaired t-test). (C) Levels of TCA metabolites downstream of α-KGDH in RZ and BZ from cs-Ttp−/− mice7 days after coronary artery ligation (n=4 mice per group, two-tailed unpaired t-test). (D,E) Summary of the OCR traces of basal (D) and maximal (E) respiration in H9c2 cells with overexpression of C124R and WT TTP (n=3 biological replicates, two-tailed unpaired t-test). (F,G) Summary of the basal (F) and maximal (G) respiration in the RZ and BZ of cs-Ttp−/− mice (n=3 biological replicates, two-tailed unpaired t-test). (H) α-KGDH activity in the BZ and RZ of WT and cs-Ttp−/− mice 7 days after coronary artery ligation (n=3 mice per group, two-tailed unpaired t-test). (I,J) NADH/NAD ratio (I) and ATP (J) levels in the RZ and BZ of and cs-Ttp−/− mice (n=3 mice per group, two-tailed unpaired t-test).

FIGS. 5A-5J. TTP deletion protects against ischemic injury through α-KGDH. (A,B) EF (A) and FS (B) of WT and cs-Ttp−/− mice 7 days after sham and coronary artery ligation (n=6 mice per condition, two-way ANOVA and Tukey's post-hoc test). (C) Schematic presentation of sections of the heart taken after MI surgery. (D) Masson's Trichrome staining of various heart sections from WT and cs-Ttp−/− mice after MI surgery. (E) Quantification of scar area from analysis of sections in Panel D (n=3 mice per condition, two-way ANOVA and Tukey's post-hoc test). (F) Schematic workflow for surgical procedure on mice treated with α-KGDH inhibitor AA6 followed by MI and sample collection. (G) Western blot analysis of LIAS protein levels in WT mice treated with sh-Control or sh-Lias AAV. HPRT1 serves as the loading control. (H) Western blot densitometry analysis of LIAS protein levels from panel (p) (n=3 biological replicates, two-tailed unpaired t-test). (I and J) EF (I) and FS (J) of WT and cs-Ttp−/− mice treated with sh-Control or sh-Lias AAV, 7 days post-MI (n=3 mice per condition, two-way ANOVA with Tukey's post-hoc test).

FIGS. 6A-6N. Infiltration of the inflammatory cells in the BZ induces TTP expression. (A) KEGG pathway enrichment of differentially expressed genes (DEGs) of RNA-seq data from the RZ and BZ after MI. The dataset supporting these findings is available at the NCBI Gene Expression Omnibus (GSE183168). (B) H&E staining of the whole heart, RZ, BZ and IZ. Arrows in the BZ demonstrate infiltration of inflammatory cells (using a 40× objective). (C) Cytokine panel analysis showing relative expression levels of various cytokines, in the RZ and BZ of WT mice 7 days after MI. (D-F) Relative mRNA expression of 111 (D), 116 (E) and Ifng (F) in the RZ and BZ of WT mice (n=3 mice per group, two-tailed unpaired t-test). (G) Correlation analysis between Ttp and Lias mRNA expression and the infiltration of IFN-γ+ T cells in the BZ at 7 days post-MI. lif30 is a marker of IFN-γ producing cells. (H) Western blot of TTP protein in adult CMs from WT and cs-Ttp−/− hearts at baseline and 3 and 6 hours after treatment with IFN-γ or IL-6 Histone 3 serves as loading control (n=2, two-tailed unpaired t-test). (I) Western blot of LIAS, Lipo-DLST, and Lipo-DLAT proteins in adult CMs from WT and cs-Ttp−/− hearts at baseline and 6 hours after treatment with IFN-γ. HPRT1 serves as the loading control. (J-L) Western blot densitometry analysis of LIAS (J), Lipo-DLST (K), and Lipo-DLAT (L) in Panel (I) (n=3 biological replicates, 5×105 cells per group, two-tailed unpaired t-test). (M) Summary of the OCR traces of basal and maximal respiration from WT mice after treatment with IFN-γ (n=3 biological replicates, two-tailed unpaired t-test). (N) Summary of the OCR traces of basal and maximal respiration from cs-Ttp−/− mice after treatment with IL-6 (n=3 biological replicates, two-tailed unpaired t-test).

FIGS. 7A-7S. Inhibition of IFN-γ rescues metabolic alterations in the BZ and protects against injury. (A) Schematic workflow for the surgical procedure on WT mice, administration of IFN-γ neutralizing antibody, and sample collection. (B) Fractional shortening (FS) in the BZ and RZ after treatment with IFN-γ neutralizing antibody (n=6 mice per group, two-tailed unpaired t-test). (C) Left ventricular internal diameter in systole (LVIDs) in the BZ and RZ of vehicle and IFN-γ neutralizing antibody-treated mice (n=6 mice per group, two-tailed unpaired t-test). (D) Left ventricular internal diameter in diastole (LVIDd) in the BZ and RZ of vehicle and IFN-γ neutralizing antibody-treated mice (n=6 mice per group, two-tailed unpaired t-test). (E) Ejection fraction (EF) in the BZ and RZ of vehicle and IFN-γ neutralizing antibody-treated mice (n=6 mice per group, two-tailed unpaired t-test). (F) Left ventricular end-systolic volume (LVESV) in the BZ and RZ of vehicle and IFN-γ neutralizing antibody-treated mice (n=6 mice per group, two-tailed unpaired t-test). (G) Left ventricular end-diastolic volume (LVEDV) in the BZ and RZ of vehicle and IFN-γ neutralizing antibody-treated mice (n=6 mice per group, two-tailed unpaired t-test). (H, I) Masson's Trichrome staining (H) of myocardial sections from vehicle and IFN-γ neutralizing antibody-treated mice and corresponding quantification of scar area (I) (n=6 mice per group, two-way ANOVA and Tukey's post-hoc test). (J) Western blot of TTP, LIAS, and Lipo-DLST proteins in the BZ and RZ of vehicle and IFN-γ neutralizing antibody-treated mice. HPRT1 serves as the loading control. (K, L) Densitometry analysis of LIAS (K) and Lipo-DLST (L) from Panel J (n=3 mice per group, two-tailed unpaired t-test). (M) Western blot of p-STAT1 and STAT1 in the BZ and RZ of vehicle and IFN-γ neutralizing antibody-treated mice. HPRT1 serves as the loading control. (N) Densitometry analysis of p-STAT1/STAT1 ratio in Panel M (n=3 mice per group, two-tailed unpaired t-test). (O) NAD+/NADH ratio in BZ from the vehicle and IFN-γ neutralizing antibody-treated mice (n=3 mice per group, two-tailed unpaired t-test). (p-s) Relative mRNA expression of Jak1 (P), Irf1 (Q), Cxcl10 (R), and Stat1 (S), in the RZ and BZ of vehicle and IFN-γ NAb-treated mice (n=6 mice per group, two-tailed unpaired t-test).

FIGS. 8A-8G. Metabolic profile of the BZ, Related to FIG. 1. (A) Western blot analysis of ANF in the BZ and RZ of the heart tissue 7 days after coronary artery ligation. GAPDH was used as a loading control (n=4 mice per group, two-tailed unpaired t-test). (B) Principal component analysis (PCA) of steady-state metabolomics data from the BZ and RZ samples collected 7 days after coronary artery ligation (n=4 mice per group). (C) Volcano plot illustrating the comparative log 2 fold changes in metabolite levels between the RZ and BZ in heart tissue 7 days after coronary artery ligation (n=4 mice per group, unpaired t-test). (D) Other selected metabolites in the BZ and RZ (n=4 mice per group, two-tailed unpaired t-test). (E) α-KGDH activity in the whole heart tissue collected from mice 7 days after coronary artery ligation (n=3 mice per group, two-tailed unpaired t-test). (F) ATP levels in the RZ and BZ (n=6 mice per group, two-tailed unpaired t-test). (G) Steady-state levels of NAD+ and NADH in the RZ and BZ (n=4 mice per group, two-tailed unpaired t-test).

FIGS. 9A-9E. mRNA levels and lipoylated proteins in BZ and RZ, Related to FIG. 2. (A) Relative mRNA expression of TCA cycle enzymes in the BZ and RZ of the heart 7 days after coronary artery ligation. (n=3 mice per group, two-tailed unpaired t-test). Cs: Citrate Synthase, Idh1: Isocitrate Dehydrogenase 1, Sdhc: Succinate Dehydrogenase Complex Subunit C, Sdhb: Succinate Dehydrogenase Complex Subunit B, Sdhd: Succinate Dehydrogenase Complex Subunit D, Mdh2: Malate Dehydrogenase 2. (B-D) Western blot of SDHB and ACO2 in the BZ and RZ of WT mice 7 days after coronary artery ligation. GAPDH was used as a loading control (n=4 mice per group, two-tailed unpaired t-test). Succinate Dehydrogenase Complex Subunit B (SDHB), ACO2: Aconitase 2. (E) Relative mRNA expression of Lias in the RZ and BZ WT and cs-Ttp−/− mice. Mouse 18S rRNA was used as the internal control for normalization (n=6 mice per group, unpaired t test).

FIGS. 10A-10N. mRNA and protein levels of the components of α-KGDH in the BZ and RZ, Related to FIG. 3. (A) Volcano plot illustrating the differentially expressed genes between the RZ and BZ in an MI mouse model, with TTP marked by an arrow. The dataset supporting these findings is available at the NCBI Gene Expression Omnibus (GSE183168). (B) Relative mRNA expression of Ttp, Dlst and Lias in the BZ and RZ WT and cs-Ttp−/− mice 7 days after coronary artery ligation. Mouse 18S rRNA was used as the internal control for normalization (n=6 mice per group, unpaired t test). (C) Western blot of LIAS, lipoylated DLST (Lipo-DLST) and DLST in the BZ ad RZ WT and cs-Ttp−/− mice 7 days after coronary artery ligation. GAPDH was used as a loading control (n=3 mice per group, two-tailed unpaired t-test). (D,E) Western blot densitometry analysis of LIAS (D) and Lipo-DLST (E) in Panel C (n=3 mice per group, two-tailed unpaired t-test). (F) Western blot of OGDH, DLST and DLD in the BZ and RZ cs-Ttp−/− mice 7 days after coronary artery ligation. GAPDH was used as a loading control (n=4 mice per group). (G) Western blot densitometry analysis of OGDH, DLST and DLD in the BZ ad RZ cs-Ttp−/− mice in Panel G (n=4 mice per group, two-tailed unpaired t-test). (H) Schematic map of AU-rich elements (AREs) in the 3′ UTR of Lias mRNA in human and mouse. SEQ ID NO: 1, SEQ ID NO: 2 (in descending order) (I) Map of AREs in the 3′ UTR of Lias mRNA in human and mouse. SEQ ID NO: 2, SEQ ID NO: 1 (in descending order) (J) Western blot of Lipo-DLAT in the BZ and RZ of WT and cs-Ttp−/− mice (n=4, two-tailed unpaired t-test). (K) Western blot densitometry analysis of Lipo-DLAT and Lipo-DLST in (L) in the BZ ad RZ of WT and cs-Ttp−/− mice in Panel (J). (M) Measurement of pyruvate catabolism in BZ ad RZ of WT mice 7 days after coronary artery ligation (n=3 mice per group, two-tailed unpaired t-test). (N) M+2 citrate: M+3 pyruvate in RZ and BZ from WT mice (n=4 mice per group, two-tailed unpaired t-test).

FIGS. 11A-11C. Metabolomic data of the BZ and RZ of cs-Ttp^{-/-} mice, Related to FIG. 4. (A) Principal component analysis of metabolomic profiles from the BZ and RZ of cs-Ttp^{-/-} mice 7 days after coronary artery ligation (n=4 mice per group). (B) Heatmap representation of steady-state metabolomics in the BZ and RZ of cs-Ttp^{-/-} mice 7 days after coronary artery ligation (n=4 mice per group). (C) Levels of select TCA cycle metabolites in H9c2 cells with overexpression of GFP control or TTP (n=3 biological replicates per condition, two-way ANOVA and Tukey's post-hoc test).

FIGS. 12A-12L. Cardiac function in cs-Ttp^{-/-} after MI and response to AA6 or and AAV containing Lias shRNA. (A) HR, EF and FS of WT and cs-Ttp−/− mice 3 days after sham surgery (n=3 mice per condition, two-way ANOVA and Tukey's post-hoc test). (B, C) MT staining of various heart sections from WT and cs-Ttp−/− mice 3 days post-MI. (D) HR of WT and cs-Ttp−/− mice 7 days after sham surgery (n=3 mice per condition, two-way ANOVA and Tukey's post-hoc test). (E) HR in WT and cs-Ttp−/− mice treated with sh-Control or sh-Lias 7 days post-MI (n=7 mice per group, two-way ANOVA and Tukey's post-hoc test). (F) Schematic workflow for AA6 treatment in WT and cs-Ttp−/− mice, showing injection and sample collection timeline. (G-I) Cardiac functional parameters, including HR (G), EF (H), and FS (I), in WT and cs-Ttp−/− mice treated with vehicle or AA6 7 days post-sham surgery (n=4 mice per group, two-way ANOVA and Tukey's post-hoc test). (J-L) Cardiac functional parameters, including HR (J), EF (K), and FS (L) in WT and cs-Ttp−/− mice treated with vehicle or AA6 7 days post-MI (n=4 mice per group, two-way ANOVA and Tukey's post-hoc test). Data are presented as mean±SEM. *P<0.05, **P<0.01, ***P<0.001, ****P<0.0001.

FIGS. 13A-13G. Levels of inflammatory markers in the BZ and RZ and the effects of inflammation on the levels of α-KGDH components, Related to FIG. 6. (A) Relative mRNA expression of Cxcl10, Hmox1 and Jak1 the BZ and RZ of WT mice 7 days after coronary artery ligation (n=4 mice per group, two-tailed unpaired t-test). Cxcl10: C-X-C motif chemokine ligand 10, Hmox1: Heme oxygenase 1, Jak1: Janus kinase 1. (B) Western blot of phosphorylated STAT1 and STAT1 in the BZ and RZ of WT mice 7 days after coronary artery ligation. GAPDH was used as a loading control (n=3 mice per group). (C) Relative mRNA expression of Ttp, Lias, Ogdh, and Dlst in H9c2 cells at different time points after treatment with IL-6 (n=3 biological replicates per condition, two-way ANOVA and Tukey's post-hoc test). (D,E) Western blot densitometry analysis of Panel (C) (n=3 biological replicates per condition, wo-way ANOVA and Tukey's post-hoc test). (F) Summary of the OCR analysis of basal and maximal respiration in isolated CMs from cs-Ttp−/− mice at baseline or after treatment with IFN-γ and siRNA against Lias (n=3 biological replicates per condition, two-tailed unpaired t-test). (G) Western blot of TTP in WT and cs-Ttp−/− hearts at baseline and after 3 and 6 hours of treatment with 50 ng/g IFN-γ. Histone H3 served as the loading control.

FIGS. 14A-14H. (A, B) Gene Set Enrichment Analysis (GSEA) plots for Interferon Gamma (IFN-γ) Response (A) and Interferon Alpha (IFN-α) Response (B) among significantly upregulated genes in the border zone (BZ). (C) Normalized Enrichment Scores (NES) for the top enriched Hallmark pathways in the BZ compared to the remote zone (RZ) in human samples. (D-H). Cardiac functional parameters in vehicle- and IFN-γ neutralizing antibody (NAb)-treated mice 7 days post-coronary artery ligation. Data include heart rate (HR, D), left ventricular anterior wall thickness in systole (LVAWs, E) and diastole (LVAWd, F), and left ventricular posterior wall thickness in systole (LVPWs, G) and diastole (LVPWd, H) (n=6 mice per group, two-tailed unpaired t-test). Data are represented as mean±SEM, with no significant differences (NS) observed between groups.

### DETAILED DESCRIPTION

The present disclosure provides methods for treating myocardial infarction. Myocardial infarction (MI) is associated with a number of metabolomic changes. While the metabolic changes in the ischemic zone (IZ) are often studied, the metabolic and remodeling changes in the areas surrounding ischemic region, known as the border zone (BZ) are unknown. The inventors demonstrate herein changes in metabolites within the BZ which suggest a block in the TCA cycle at the level of the enzyme α-KGDH, and an increase in production of tristetraprolin (TTP) in cardiomyocytes of the BZ. Mice with cardiac-specific deletion of TTP (cs-Ttp−/−) showed no block at the level of α-KGDH in the BZ, and were protected against ischemic injury, which was reversed by the inhibition of α-KGDH activity. Finally, the inventors demonstrate that inflammatory cytokines in the BZ are responsible for the increase in TTP and subsequent inhibition of α-KGDH activity, and that neutralization of interferon-γ improves cardiac function after ischemic injury. These studies demonstrate that inflammation in the BZ has deleterious effects on cardiomyocytes by inhibiting α-KGDH through TTP, resulting in adverse remodeling after myocardial infarction. Additionally, the inventors demonstrate that targeting interferon-γ signaling may be a viable strategy for cardiac protection after MI.

One aspect of the present disclosure provides a method of treating myocardial infarction (MI) in a subject in need, the method comprising administering to the subject a therapeutically effective amount of at least one of an interferon-gamma (IFNγ) inhibitor and an interleukin 6 (IL-6) inhibitor.

Myocardial infarction (MI), colloquially known as “heart attack,” is caused by decreased or complete cessation of blood flow to a portion of the myocardium. Myocardial infarction may be “silent,” and go undetected, or it could be a catastrophic event leading to hemodynamic deterioration and sudden death. Most myocardial infarctions are due to underlying coronary artery disease. With coronary artery occlusion, the myocardium is deprived of oxygen. Prolonged deprivation of oxygen supply to the myocardium can lead to myocardial cell death and necrosis. Patients can present with chest discomfort or pressure that can radiate to the neck, jaw, shoulder, or arm. In addition to the history and physical exam, myocardial ischemia may be associated with ECG changes and elevated biochemical markers such as cardiac troponins. Different imaging techniques are used to assess myocardial perfusion, myocardial viability, myocardial thickness, thickening and motion, and the effect of myocyte loss on the kinetics of para-magnetic or radio-opaque contrast agents indicating myocardial fibrosis or scars. Some imaging modalities that can be used are echocardiography, radionuclide imaging, and cardiac magnetic resonance imaging (cardiac MRI). Treatments for MI include, but are not limited to reperfusion therapy, pain relief, nitrates, beta blockers, platelet inhibition, lipid lowering treatments, antithrombotic therapy, surgery and lifestyle modifications.

Interferons (IFNs) are proteins that belong to the group of signaling molecules known as cytokines involved in the upregulation of the immune response. Interferons are particularly important in fighting viral infections but also play a vital role in tumor suppression, upregulation of MHC Class 1 and 2, signal transduction, and activation of immune cells, including natural killer cells and macrophages. There are three main types of interferons including interferon-alpha, interferon-beta, and interferon-gamma. Interferon-alpha and interferon-beta are Type 1 (I) interferons, interferon-gamma is a Type 2 (II) interferon and interferon lambda is a third subclass, Type 3 (III). Interferons exhibit immune-modulatory effects by initiating signaling cascades that lead to the expression of gene products such as MHC class 1, B2 microglobulin, and others. Interferons induce the expression of hundreds of genes, which mediate various biological responses. Some genes are regulated by both type I and type II (and/or type III), while others are selectively regulated by distinct IFNs. IFNs activate the JAK-STAT pathways, which includes the nuclear translocation and initiation of gene transcription by STATs that have been activated at the plasma membrane in response to JAK-mediated phosphorylation. IFNs have also been shown to activate other signaling pathways, including MAP Kinase and PI3K signaling.

IFN-gamma is the only known type II interferon. While it does not share structural homology or a common receptor with the type I IFNs, it too has antiviral and immunomodulatory properties. The biologically active form of IFN-gamma is a noncovalently-linked homodimer. This homodimer binds to the extracellular domain of two IFN-gamma R1/CD119 chains, which interact with IFN-gamma R2 to form the functional IFN-gamma receptor complex. The IFN-gamma R1 subunits of the receptor complex are associated with Jak1, while the IFN-gamma R2 subunits are associated with Jak2. Activation of Jak1 and Jak2 results in phosphorylation of the receptor and subsequent recruitment and phosphorylation of STAT1. STAT1 phosphorylation leads to its homodimerization and nuclear translocation. Once in the nucleus, STAT1 homodimers bind to IFN-gamma-activated sequence (GAS) elements in the promoters of target genes to regulate their transcription. Many of the target genes that are induced by IFN-gamma/STAT1 signaling are transcription factors that then drive the expression of secondary response genes. In addition, IFN-gamma signaling can activate MAPK, PI 3-K-Akt, and NF-kappa B signaling pathways to regulate the expression of a number of other genes. IFN-gamma signaling plays a key role in host defense by promoting macrophage activation, upregulating the expression of antigen processing and presentation molecules, driving the development and activation of Th1 cells, enhancing natural killer cell activity, regulating B cell functions, and inducing the production of chemokines that promote effector cell trafficking to sites of inflammation. While IFN-gamma has historically been known for its cytotoxic, cytostatic, and anti-tumor properties, multiple studies have also suggested that IFN-gamma may also have context-dependent proliferative and pro-tumorigenic effects.

Type II interferon signaling in cardiomyocytes refers to the effects of IFN-γ, on cardiac muscle cells, where it can influence various cellular processes including inflammation, fibrosis, and potentially contribute to the development of heart disease through its interaction with specific receptors on the cardiomyocyte surface, often leading to changes in gene expression and cellular behavior.

As used herein, a “interferon-gamma inhibitor” refers to any compound or molecule that is capable of inhibiting the action of, and/or reducing the expression and/or function of type II interferon (IFNγ) or IFNγ signaling. In some embodiments, the inhibitor is selected from the group consisting of antibodies, small molecules, peptides, miRNAs, siRNAs, oligonucleotides, genome editors, protein degraders, cytokines, agonists and combinations thereof. In some embodiments, the inhibitor is selected from the group consisting of Emapalumab, Ruxolitinib, Jak/Stat inhibitors and combinations thereof.

Interleukin-6 (IL-6) is a pro-inflammatory cytokine secreted by macrophages in response to stress, infection, or injury. The term “interleukin-6 inhibitor” refers to any compound or molecule that is capable of inhibiting the action of, and/or reducing the expression and/or function of IL-6 signaling. Suitable IL-6 inhibitors for use in the methods described herein include, but are not limited to, Tocilizumab, Sarilumab, and Siltuximab.

The terms “antibody” and “antibody molecule” are used herein interchangeably and refer to immunoglobulin molecules or other molecules which comprise an antigen binding domain. Antibodies include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g. ability to bind to the protein of interest or variant) are retained.

As used herein, the terms “small molecular therapeutic agent”, “small molecule compound”, and “small molecule drug” refer to a chemical compound or pharmaceutically acceptable salt thereof having a therapeutic effect and/or enhancing the therapeutic effect of an immunotherapy. Small-molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

The gene silencing agent may comprise a nucleic acid sequence that is capable of inducing RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Two types of small RNA molecules may induce RNAi: microRNA (miRNA) and small interfering RNA (siRNA). miRNA is a small non-coding RNA molecule (typically containing about 20-25 nucleotides) found in plants, animals and some viruses, which silences complementary target sequences by one or more of the following processes: (1) cleavage of the target mRNA strand into two pieces, (2) destabilization of the mRNA through shortening of its poly(A) tail, and (3) less efficient translation of the mRNA into proteins by ribosomes. (See Bartel D. P., Cell, 136 (2): 215-233 (2009); and Fabian et al., Annual Review of Biochemistry, 79: 351-79 (2010)). siRNA (also known as short interfering RNA or silencing RNA), is a class of double-stranded RNA molecules, typically 20-25 base pairs in length, which silence complementary target sequences by degrading mRNA after transcription, preventing translation. (See Dana et al., International Journal of Biomedical Science, 13(2):48-57 (2017); and Agrawal, et al., Microbiol. Mol. Biol. Rev., 67: 657-685 (2003)). siRNA can also act in RNAi-related pathways in an antiviral mechanism or play a role in the shaping of the chromatin structure of a genome. Any RNA molecule that is capable of silencing gene expression of a target gene may be used in connection with the present disclosure. In some embodiments, the RNA molecule is siRNA, miRNA, antisense oligos. In other embodiments, the RNA molecule may a long non-coding RNA (lncRNA). Long non-coding RNAs are a large and diverse class of transcribed RNA molecules with a length of more than 200 nucleotides that do not encode proteins. lncRNAs are thought to encompass nearly 30,000 different transcripts in humans, and account for the major part of the non-coding transcriptome. While the mechanism of action of lncRNAs is under investigation, lncRNAs appear to be important regulators of gene expression, and lncRNAs are thought to have a wide range of functions in cellular and developmental processes. lncRNAs may carry out both gene inhibition and gene activation through a range of diverse mechanisms (see, e.g., Kung et al., Genetics, 193(3): 651-666 (2013); and Marchese et al., Genome Biol., 18: 206 (2017)).

The terms “protein” or “polypeptide” or “peptide” are used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

Therapeutic cytokines are engineered proteins that mimic the body's own signaling molecules to modulate the immune system.

Protein degraders are drugs that use the body's own cellular machinery (such as the proteasome or lysosomes) to tag and destroy disease-causing proteins, rather than just blocking them. Protein degraders include PROTACs (Proteolysis-Targeting Chimeras), which act as molecular bridges, bringing a target protein to an E3 ligase, which then flags the protein for breakdown.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of a myocardial infarction. The subject may be responsive to one or both of an IFNγ inhibitor and an IL-6 inhibitor. The term “treat” further includes the reduction of one or more symptoms associated with myocardial infarction, such as cardiac remodeling, myocyte damage or death, inflammation, ischemic injury, increased angiogenesis, and/or fibrosis. Treating the MI may result in reduced damage or death of cardiomyocytes, decreased fibrosis, improved cardiac function, improved cardiac structure, reduced chamber dilation, and/or improved contractile function. Cardiac function may be evaluated with electrocardiograms (ECG), echocardiograms, and coronary angiography, blood tests, evaluation of cardiac enzymes like troponin. Treating the MI may also refer to molecular changes, for example in the infarct zone or border zone. Molecular changes may include, but are not limited to, decreased cytokines, decreased TTP, and/or decreased levels of Stat1, Jak1, Irf1 and Cxcl10. Additionally, treatment of MI may include reducing or delaying inflammation in the border zone of an MI. Inflammation in the border zone of an MI may be characterized by the production or presence of cytokines such as IL-1β, IL-6, IFNγ; other inflammatory related proteins such as CXCL10, Hmox1, Jak1, STAT1; or inflammatory cells such as monocytes, macrophages, neutrophils, dendritic cells, and or T and B lymphocytes.

As used herein the terms “therapeutically effective amount” and “effective amount” refer to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be improving cardiac function after MI.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

As used herein, the term “administering” an agent, such as a therapeutic entity described herein to an subject, animal or cell, is intended to refer to dispensing, delivering or applying the agent to the intended target. The agent may be administered by any suitable route for delivery of the therapeutic agent to the desired location in the animal, including, but not limited to, delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

Any of the IFNγ inhibitors and IL-6 inhibitors described herein may be formulated in a pharmaceutical composition further comprising a pharmaceutically acceptable carrier. The term “pharmaceutically acceptable carrier” as used herein means a non-toxic, inert liquid filler, diluent, encapsulating material or formulation auxiliary of any type. The pharmaceutical compositions may be formulated for administration by, for example, injection. Therapeutic compositions typically are sterile and stable under the conditions of manufacture and storage. The pharmaceutically acceptable carrier used in the compositions described herein may be a diluent to dilute the ethanol to the proper percentage, such as sterile water, phosphate buffer saline, and the like. Additional suitable pharmaceutically acceptable carriers are known in the art, and include, but are not limited to, diluents, preservatives, solubilizers, emulsifiers, liposomes, nanoparticles and adjuvants, buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate or other carriers, according to the judgment of the formulator. Additionally, pharmaceutically acceptable carriers may be aqueous or non-aqueous solutions, suspensions, and emulsions. Examples of aqueous carriers include isotonic solutions, alcoholic/aqueous solutions, emulsions or suspensions, including saline and buffered media. Techniques, formulations, and pharmaceutically acceptable carriers may generally be found in Alphonso Gennaro, ed., Remington's Pharmaceutical Sciences, 18th Ed., (1990) Mack Publishing Co., Easton, Pa.

A “subject in need” as utilized herein may refer to a subject in need of treatment for myocardial infarction. The term “subject” may be used interchangeably with the terms “individual” and “patient” and includes human and non-human mammalian subjects. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like.

In the example provided herein, the IFN-γ neutralizing antibody was administered via intraperitoneal (IP) injection at a dosage of 50 ng/kg. This method was chosen for its reliability and consistency in delivering the antibody systemically. While intraperitoneal administration was used in this study, alternative routes, such as intravenous (IV) or subcutaneous (SC), may also be used, depending on the therapeutic application and desired pharmacokinetics. Further additional dosing schedules may be used for example a range in the number of doses and the concentration of the dose in addition to the route of administration. In some embodiments, about 25 ng/kg to about 50 ng/kg and any value in-between may be administered. In some embodiments, about 50 ng/kg, about 45 ng/kg, about 40 ng/kg, about 35 ng/kg, about 30 ng/kg, and about 25 ng/kg or any value in-between may be administered. In embodiments, about 50 ng/kg IFNγ is administered.

The IFNγ inhibitor and IL-6 inhibitor may be administered at least once following an MI. In embodiments, the inhibitor is administered no more than twice to treat the MI. In some embodiments, an IFNγ inhibitor and/or IL-6 inhibitor is administered 1 time, 2 times, 3, times, 4 times, 5 times, 6 times, 7 times, 8 times, 9 times 10 times or more following an MI. The IFNγ inhibitor and/or IL-6 inhibitor may be administered when a MI is suspected or when a subject has symptoms of a myocardial infarction. Symptoms may include, but are not limited to chest pain or discomfort, discomfort in other areas of the upper body, shortness of breath, breaking out in cold sweats, nausea or light-headedness, backpain, dizziness, fainting, pressure or squeezing sensation in chest, neck, shoulder or jaw, fatigue, and or vomiting. The IFNγ inhibitor and/or IL-6 inhibitor may be administered to a subject at risk for a myocardial infarction such as those with diabetes, a family history of MI, high blood cholesterol or high blood pressure.

The IFNγ inhibitor and/or IL-6 inhibitor may be administered about one day after a MI. In some embodiments, the IFNγ inhibitor and/or IL-6 inhibitor is administered about one day after an MI, and again 1, 2, 3, 4, 5, 6, 7, 8, 9, 10, 15, 20 or more days later.

In embodiments, the method further comprising one or more additional therapies. Additional therapies may include, but are not limited to anticoagulants, antianginals, narcotics, beta blockers, statins, ACE inhibitors, surgery, stents, angioplasty and combinations thereof.

In certain embodiments, the one or more additional therapies are administered according to standard clinical practice and may be delivered prior to, concurrently with, or following administration of the interferon-gamma inhibitor. The additional therapies may be administered as a single dose or as repeated doses over an acute, subacute, or chronic treatment period. In some embodiments, dosing may occur once daily, multiple times per day, or at intervals ranging from hours to weeks, depending on the clinical condition of the subject.

Another aspect of the present disclosure provides a method of reducing damage or death of cardiomyocytes, the method comprising administering an IFNγ inhibitor and or an IL-6 inhibitor. The cardiomyocytes may be damaged or dying as a result from a lack of oxygen or in associated with a MI. In some embodiments, the cardiomyocytes are in an infarct zone or in a border zone. Cardiomyocytes are the contractile myocytes of the cardiac muscle. Each cardiomyocyte contracts in coordination with neighboring cells to pump blood from the heart. Cardiomyocyte damage and death may be evaluated based on biomarkers including but not limited to cardiac troponin (e.g. troponin-I or troponin-T), heart type fatty acid binding protein (H-FABP), myosin light chain 1 (MHLC-1), and creatine kinase-MB; and via cardiac magnetic resonance, and microscopy measurements such as TUNEL and DNA laddering.

Protein, DNA or RNA expression may be measured, detected or quantitated by any means known in the art. Examples include, but are not limited to spectroscopic methods, immunoassays (ELISA, Western blot, flow cytometry, immunochemistry), high-throughput methods, Q=quantitative PCR (qPCR)/RT-qPCR, northern blotting, and sequencing. Protein activity may also be measured by any means known in the art, including, but not limited to enzyme assays, binding assays, immunoassays, signaling assays and cell-based assays.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this disclosure are described herein, including the best mode known to the inventors for carrying out the disclosure. Variations of those preferred aspects may become apparent to those of ordinary skill in the art upon reading the foregoing description. The inventors expect a person having ordinary skill in the art to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The following examples are meant only to be illustrative and are not meant as limitations on the scope of the disclosure or of the appended claims.

### EXAMPLES

The following Examples are illustrative and should not be interpreted to limit the scope of the claimed subject matter.

### Example 1—Distinct Inflammatory and Metabolic Features of the Border Zone Define

### Therapeutic Targets Following Myocardial Infarction

### Nonlimiting List of Abbreviations Used in this Example

1. MI—Myocardial Infarction
2. BZ—Border Zone
3. RZ—Remote Zone
4. IZ—Infarct Zone
5. TCA—Tricarboxylic Acid
6. α-KGDH—Alpha-Ketoglutarate Dehydrogenase
7. LIAS—Lipoic Acid Synthase
8. DLST—Dihydrolipoamide S-Succinyltransferase
9. DLD—Dihydrolipoamide Dehydrogenase
10. OGDH—Oxoglutarate Dehydrogenase
11. TTP—Tristetraprolin
12. OCR—Oxygen Consumption Rate
13. IFN-γ—Interferon Gamma
14. IL—Interleukin
15. CXCL10—C-X-C Motif Chemokine Ligand 10
16. Hmox1—Heme Oxygenase 1
17. Jak1—Janus Kinase 1
18. STAT1—Signal Transducer and Activator of Transcription 1
19. EF—Ejection Fraction
20. FS—Fractional Shortening
21. AA6—(S)-2-[(2,6-dichlorobenzoyl) amino] succinic acid
22. shRNA—Short Hairpin RNA
23. AAV—Adeno-Associated Virus
24. PCR—Polymerase Chain Reaction
25. RNA-seq—RNA Sequencing
26. ARE—AU-Rich Elements
27. TZF—Tandem CCCH Zinc Fingers
28. AcD—Actinomycin D
29. STEMI—ST-Elevation Myocardial Infarction
30. NSTEMI—Non-ST-Elevation Myocardial Infarction

MI has been shown to lead to a number of metabolic changes in the heart. For example, succinate is a shown to be a metabolic signature of ischemia and to be responsible for the production of reactive oxygen species (ROS) by the mitochondria during reperfusion injury. The reason for the increased succinate is due to the reversal of succinate dehydrogenase (SDH) during ischemia, but upon reperfusion, succinate is rapidly oxidized by SDH to generate ROS from complex I (15). Another study suggested that succinate is generated mostly by canonical TCA cycle activity during ischemia, and not by SDH reversal (16). A recent study showed that inhibition of fatty acid oxidation in CMs improves r CM regeneration after ischemia-reperfusion (I/R) injury, which was associated with α-KG accumulation and activation of the α-KG dependent KDM5 (17). These results highlight the importance of the TCA cycle intermediates and enzymes in the regulation of cardiac physiology and its response to ischemic insult.

TTP, also known as Zfp36, Nup475, GOS24, and TIS11, belongs to a family of proteins containing tandem CCCH zinc fingers (TZF) (18). It binds to AU-rich elements (ARE) in the 3′ untranslated region (UTR) of certain mRNAs, promoting their degradation. TTP was originally discovered in 1990 to be stimulated by insulin (19), however, TTP knockout (KO) mice display systemic inflammation (20). The mechanism for this phenotype is through TTP binding to the AREs in the 3′ UTR of the tumor necrosis factor-α (TNFα) mRNA and promoting its degradation (21, 22). The TTP KO mice lack this regulation of TNFα, resulting in an increase in TNFα levels and systemic inflammation. Furthermore, TTP plays a role in global reprogramming of cellular iron utilization by suppressing multiple iron-consuming pathways and sparing the limited iron for essential functions (23). Additional studies suggest a link between TTP and metabolism (19, 24-27).

Inflammation plays both reparative and detrimental roles in MI (28). Pro-inflammatory cytokines such as IL-1, IL-6, and TNF-α are rapidly upregulated post-MI and play a role in clearing dead tissue; however, if uncontrolled, they can exacerbate myocardial injury and contribute to adverse remodeling (29). Anti-inflammatory therapies, including IL-1β inhibitors (e.g., canakinumab) and IL-6 antagonists (e.g., tocilizumab), have shown promising effects in reducing residual inflammatory risk and improving patient outcomes (30, 31). Additionally, inhibition of interferon (IFN) signaling using IFN regulatory factor 3 knockout (Irf3^{-/-}) mice ameliorates post MI injury, suggesting that type 1 IFN response may be a possible therapeutic avenue for protection after MI (32). However, the role of IFN-γ (i.e., type 2 interferon) in this process is not well known. Despite this, these treatments face challenges in selectively targeting harmful inflammation without disrupting critical healing processes (31). While clinical trials have demonstrated success in reducing cardiovascular events and improving myocardial salvage, broad anti-inflammatory strategies risk impairing tissue repair, leading to suboptimal outcomes (33, 34). The key objective in using these therapies is to strike a balance between controlling inflammation and promoting tissue regeneration, thereby improving outcomes for MI patients by mitigating inflammation's harmful effects while preserving its reparative functions.

Here, we studied the metabolic changes that occur in the border zone (BZ) after MI. We demonstrate that there is blockage in α-KGDH activity in the CMs of the BZ. This is due to the higher levels of TTP production in CMs of the BZ, which binds to and inhibits lipoic acid synthase (LIAS), resulting in reduced lipoylation of the E2 component of the α-KGDH complex. This leads to the inhibition of the α-KGDH activity and reduced electron transport chain activity as assessed by oxygen consumption rate (OCR). We also demonstrate that mice with cardiac specific deletion of TTP (cs-Ttp^{-/-}) mice do not display reduced α-KGDH activity and are protected against ischemic damage, which is reversed with α-KGDH inhibitors. Finally, we demonstrate that the production of TTP in the BZ is due to the activity of cytokines from infiltration of inflammatory cells into the region. These results highlight the importance of the TCA cycle enzymes in the remodeling of the heart, in addition to their role in ROS production ischemic damage in the IZ through SDH (35).

### Results

### Metabolic Analysis of the BZ Displays a Block at the Level of α-KGDH

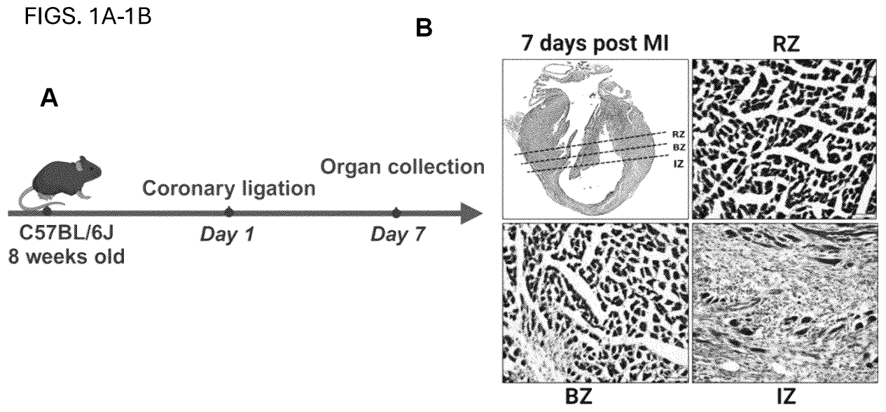

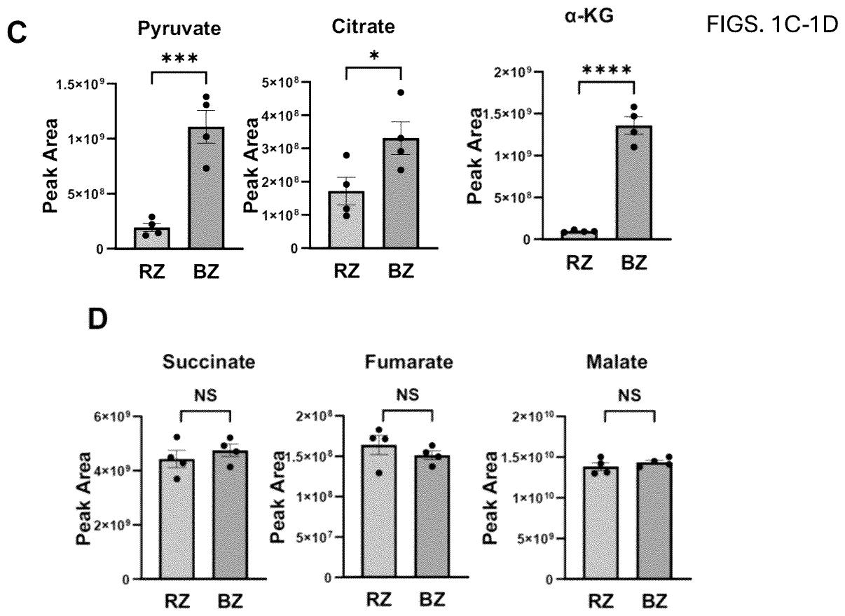

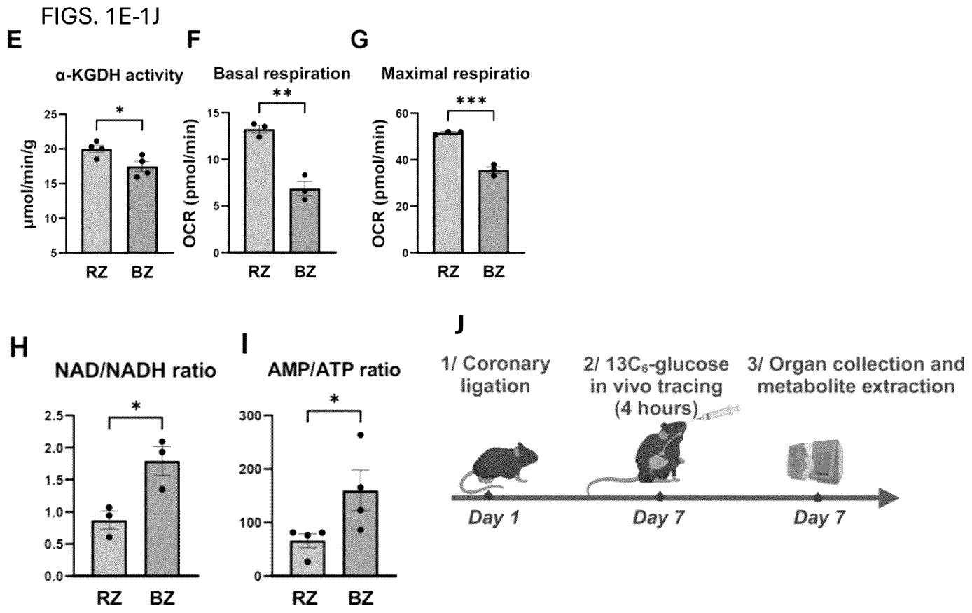

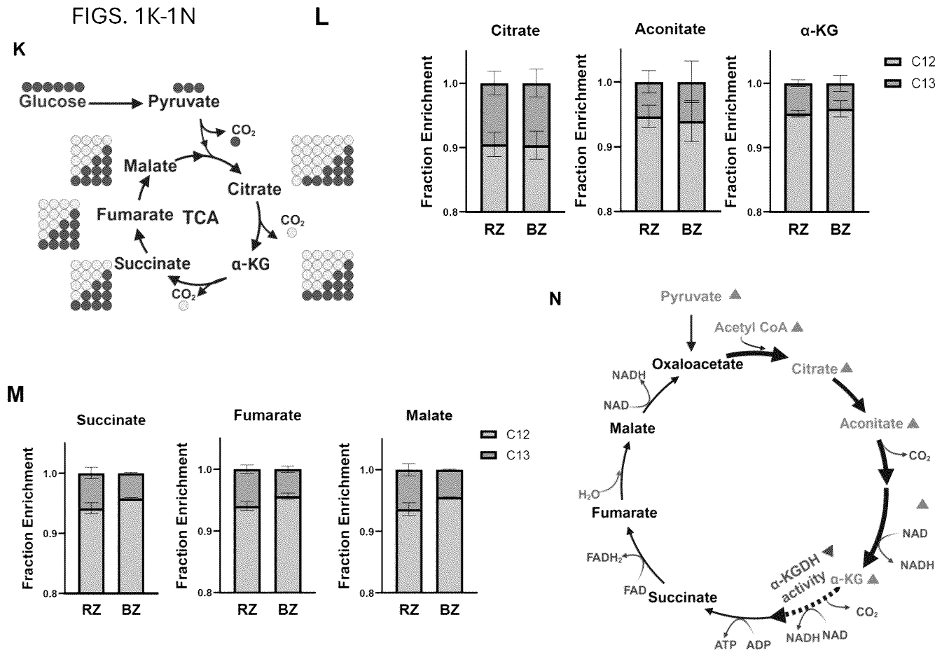

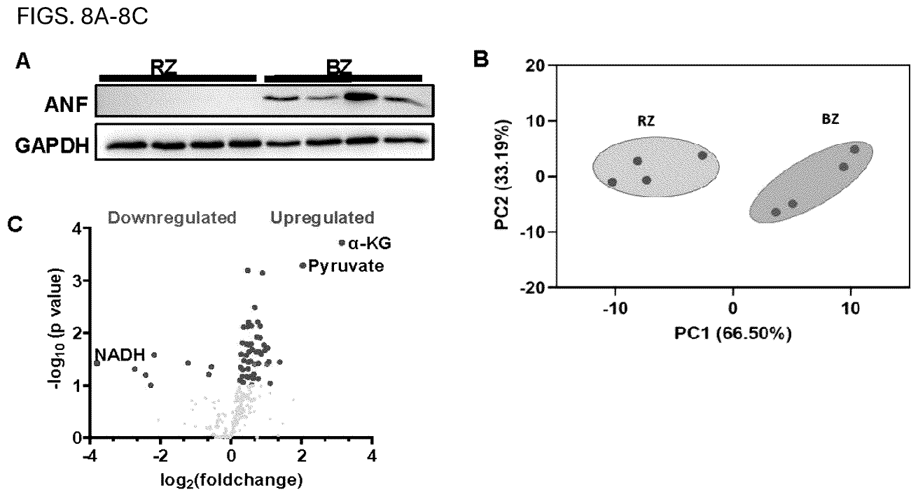

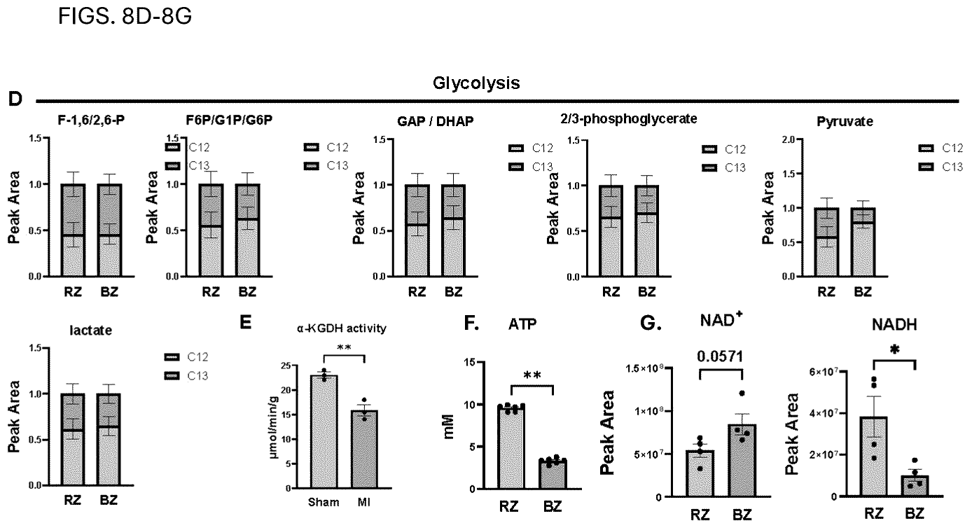

The BZ of an infarcted region plays an important role in cardiac remodeling and unlike the ischemic zone (IZ), it can be targeted to reduce the deleterious consequences of MI. Although some studies have evaluated the metabolic changes that occur in the heart in response to ischemia, it is not known how BZ around an infarcted area alters its metabolism during the remodeling process. To address this question, we subjected 8-week-old C57 mice to MI and the BZ, IZ and remote zone (RZ) samples were isolated 7 days after MI (FIG. 1A). Masson's trichrome staining (MT) confirmed the expected changes, including fibrosis, in these regions (FIG. 1). The separation of BZ and RZ was confirmed by measurement of the atrial natriuretic factor (ANF), which was higher in the BZ (FIG. 8A), as reported previously (36). To assess the metabolic profile of the regions around infarcted zone, we performed steady-state metabolomics on the BZ and RZ. IZ was not included in this analysis since myocardium in this region is mostly replaced by fibrotic tissue. Principal component (PC) analysis showed that the BZ and RZ samples cluster separately, confirming that these two populations have distinct metabolic profiles (FIG. 8B). Analysis of the metabolomic data revealed significant changes in several metabolite in the BZ, specifically a significant increase in metabolites upstream of α-KGDH, including pyruvate, citrate, and α-KG, with α-KG and pyruvate displaying the highest increase (FIG. 1C and FIG. 8C). There was no change in the metabolites downstream of α-KGDH (FIG. 1D), indicating that there is a block at the level of α-KGDH in the BZ. We also performed ¹³C-glucose tracing and noted small changes in the glycolytic flux that did not follow a pattern, and likely do not represent a change in the activity of glycolysis or any of its side branches (FIG. 8D).

To confirm the block at the α-KGDH level, we also measured α-KGDH activity in total tissue extracts from the heart after MI and after sham surgery and in the BZ and RZ and demonstrated reduced α-KGDH after MI (FIG. 8E) and in the BZ (FIG. 1E). Additionally, we measured oxygen consumption rate (OCR) in the BZ and RZ and noted a significantly lower OCR in the BZ (FIG. 1F, G), consistent with lower TCA cycle activity. We also found that NAD/NADH levels are higher and ATP levels are lower in the BZ compared to RZ (FIG. 1H,I), consistent with reduced energy generation in the BZ.

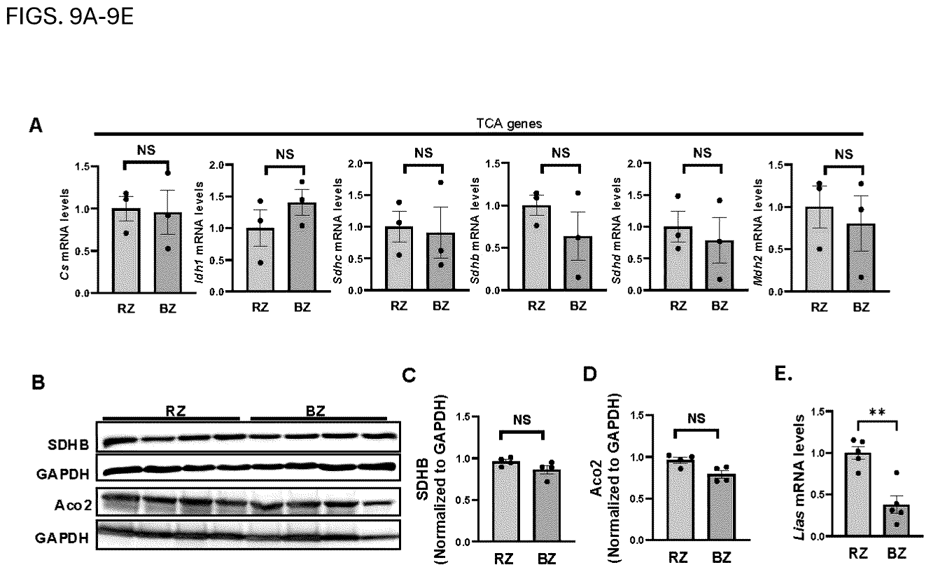

We next performed in vivo tracing experiments, by performing coronary ligation and feeding mice [¹³C₆]-glucose7 days later, followed by isolation of the BZ and RZ after 4 hours (FIG. 1J). This approach allows us to determine the rate of carbon incorporation into the TCA intermediates (FIG. 1K). These studies demonstrated that although intermediates upstream of α-KGDH showed no difference in labeling (FIG. 1L), metabolites downstream of this step (i.e., succinate, fumarate and malate) showed significantly reduced glucose labeling in the BZ (FIG. 1M), further supporting a block at the level of α-KGDH. We also measured mRNA levels of several proteins involved in TCA cycle and noted no difference in their levels in the BZ vs RZ (FIG. 9A). Additionally, levels of two major proteins in the TCA cycle, the B-subunit of succinate dehydrogenase (SDH) and aconitase (Aco2) were not altered in the BZ compared to RZ (FIG. 9B-D), indicating that the metabolite changes noted in the BZ are not due to altered levels of the enzymes in glycolysis or TCA cycle. Collectively, these results demonstrate that the BZ around an infarcted region display a blockage in the TCA cycle at the level of α-KGDH (FIG. 1N).

### The BZ Displays Reduced LIAS Levels and Lipoylation of DLST

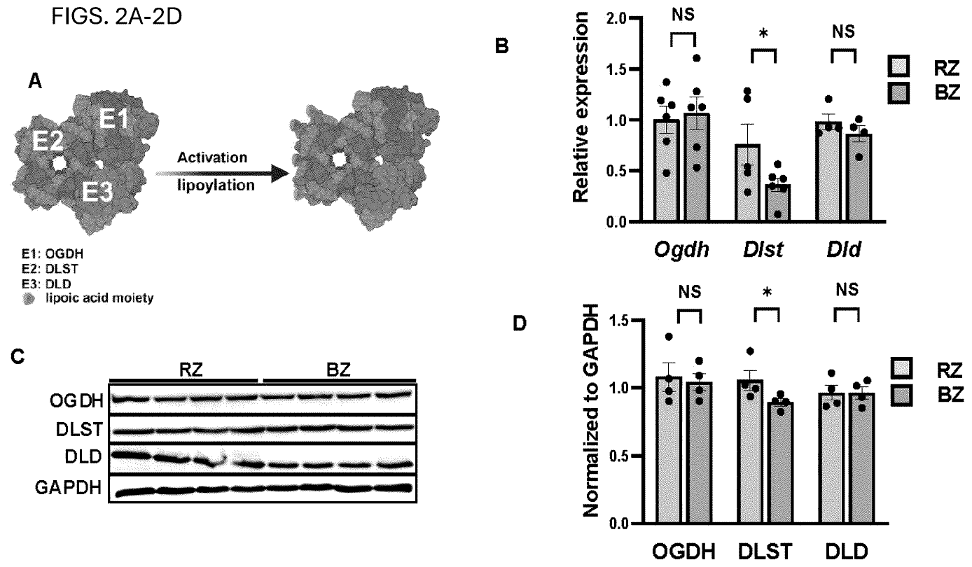

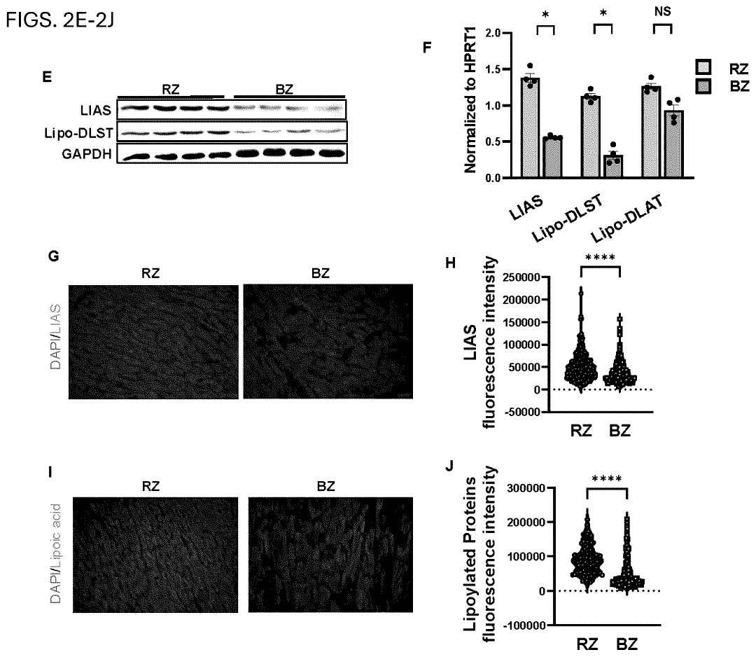

α-KGDH is a complex of OGDH (E1 subunit), DLST (E2 subunit) and DLD (E3 subunit) and its activity is regulated by lipoylation of the DLST component (FIG. 2A). To determine the mechanism by which α-KGDH activity is reduced in the BZ, we measured the mRNA and protein levels of the components of the α-KGDH complex. Although there was a reduction in the DLST mRNA (FIG. 2B), the protein levels of the three components were not different in the BZ compared to RZ (FIG. 2C,D). Since DLST needs to be lipoylated to become catalytically active, we also assessed the protein levels of the key enzyme in lipoylation, i.e., lipoic acid synthase (LIAS) and lipoylated DLST (Lipo-DLST) in the BZ and RZ. These studies demonstrated that both LIAS and Lipo-DLST are significantly reduced in the BZ (FIG. 2E,F), indicating inhibition of LIAS and the resultant DLST lipoylation in the BZ.

To confirm these results, we also assessed the levels of LIAS, total lipoylated proteins and Lipo-DLST in the BZ of infarcted hearts from patients who died within 2 weeks after ST-elevation MI (STEMI) (Table 1). These results demonstrated that LIAS (FIG. 2H) and lipoylated proteins (FIG. 2I) are significantly reduced in the BZ after STEMI compared to the RZ, supporting the data obtained in mice. α-KGDH needs to be lipoylated by DLST, DLST is lipoylated to be active by LIAS; LIAS and lipoylated proteins are reduced.

TABLE 1
Summary of clinical histories and cardiovascular
pathology of human myocardial infarction cases

| Patient | | | | Medical | Cause of | Cardiovascular | Additional |
|---|---|---|---|---|---|---|---|
| ID | MRN | Age | Sex | History | Death | Findings | Findings |
| NMA2 | 007599329 | 67 | Female | History of | Acute | Severe | Pulmonary |
| 3-00059 | | | | total | myocardial | coronary | congestion, |
| | | | | abdominal | infarction | artery | emphysemato |
| | | | | hysterectomy | (STEMI) | disease | us change, |
| | | | | | | (LAD | focal lung |
| | | | | | | complete | hemorrhage, |
| | | | | | | occlusion, | Right thyroid |
| | | | | | | LCx 50%, | nodules, |
| | | | | | | RCA 50%), | Liver |
| | | | | | | Acute MI | hemangioma, |
| | | | | | | aged 3-4 | Transient |
| | | | | | | days, Left | ischemic |
| | | | | | | ventricular | brain injury |
| | | | | | | dilation | |
| NMA2 | 111015732035 | 67 | Male | Hypothyroidism, | Acute | Acute and | Patchy |
| 3-00111 | | | | polysubstance | myocardial | recent MI | interstitial |
| | | | | abuse, recent | infarction | involving | fibrosis and |
| | | | | NSTEMI, | (NSTEMI) | anterior, | chronic |
| | | | | coronary | | lateral, and | inflammation |
| | | | | artery bypass | | posterior left | in lung, |
| | | | | grafting | | ventricle | Liver with |
| | | | | | | walls, | macrovesicul |
| | | | | | | Multifocal | ar steatosis, |
| | | | | | | remote MIs, | Small thyroid |
| | | | | | | Left | with |
| | | | | | | ventricular | lymphocytic |
| | | | | | | hypertrophy | thyroiditis, |
| | | | | | | | Adrenomegaly |
| NMA2 | N/A | 67 | Female | None | Acute | 90% LAD | Infarct in |
| 4-0038 | | | | reported | myocardial | stenosis, | interventricular |
| | | | | | infarction | Acute | septum, |
| | | | | | (STEMI) | necrosis with | Post infarct in |
| | | | | | | full spectrum | posterior wall |
| | | | | | | of histologic | |
| | | | | | | changes | |
| | | | | | | including | |
| | | | | | | myocytolysis | |
| | | | | | | and PMN | |
| | | | | | | debris in | |
| | | | | | | anterior wall, | |
| | | | | | | Acute infarct | |
| | | | | | | in lateral | |
| | | | | | | wall | |

### TTP is Increased in the BZ and Reduces DLST and LIAS Protein Levels

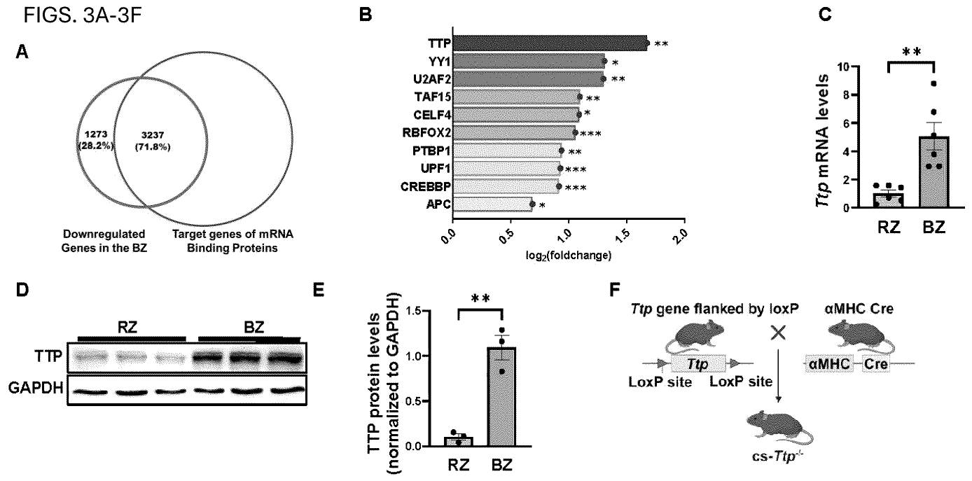

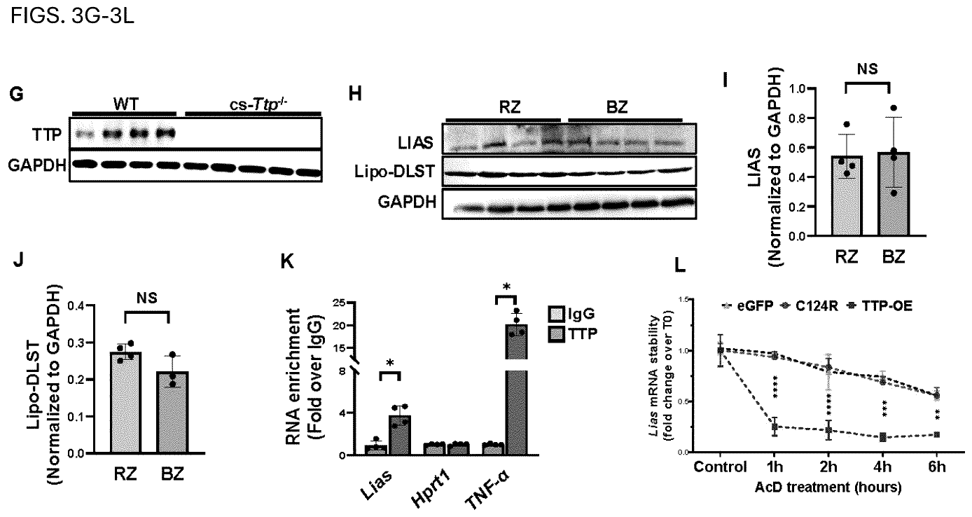

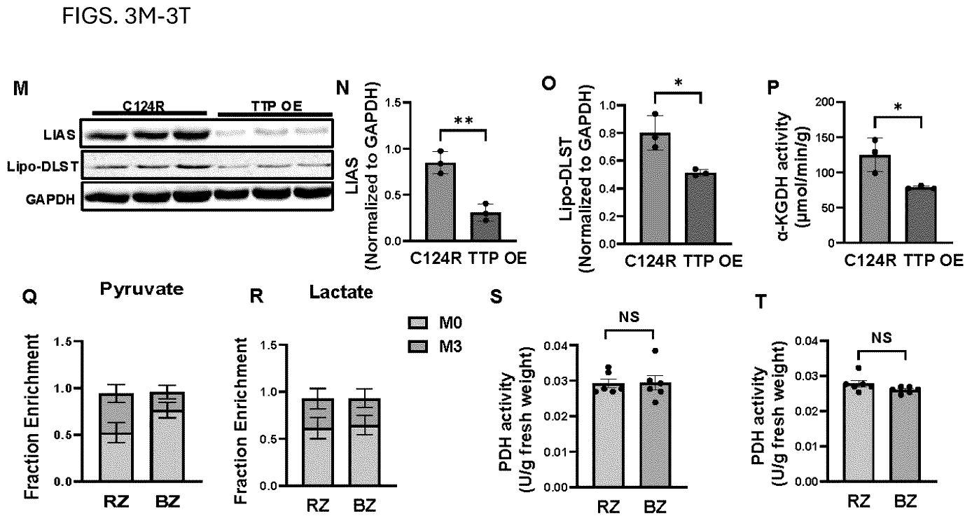

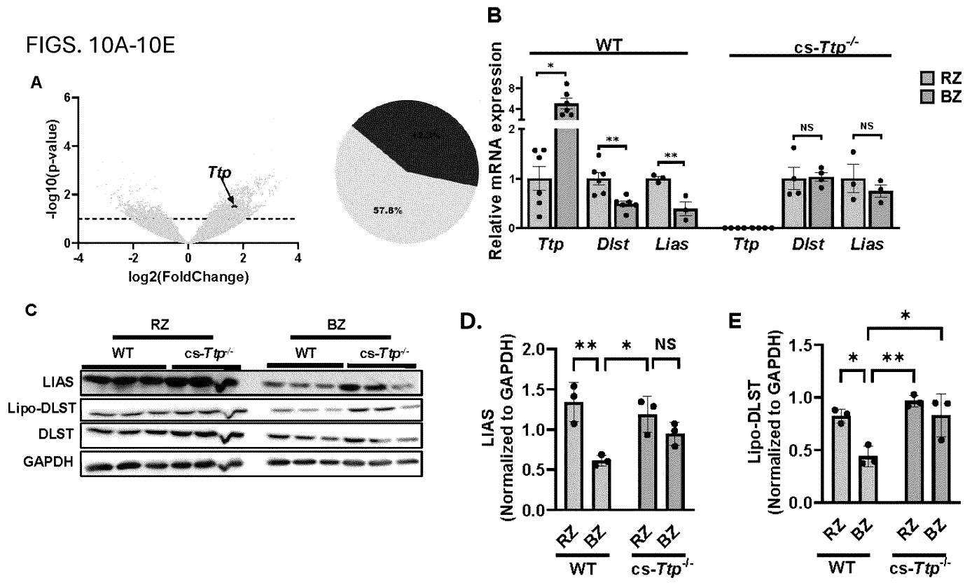

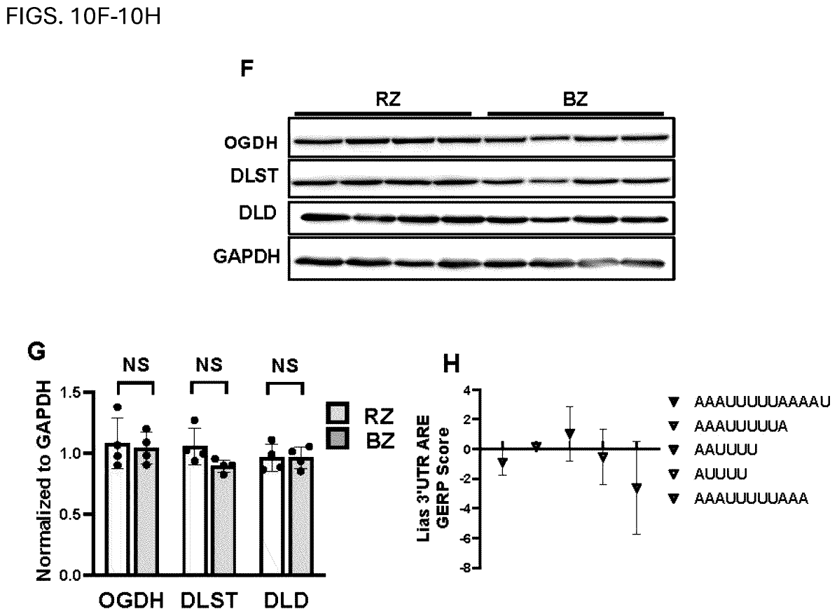

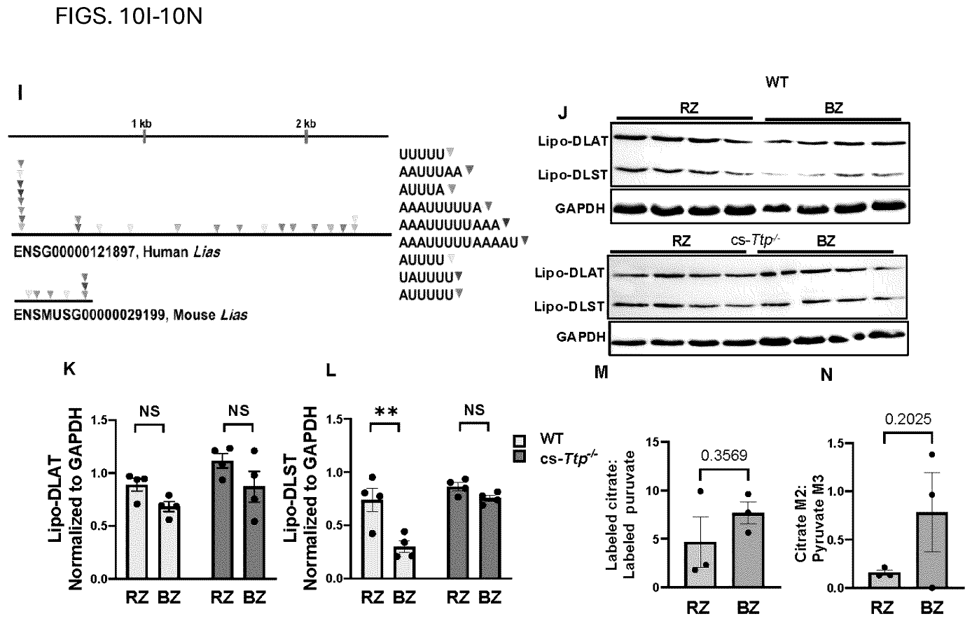

We next studied the mechanism by which α-KGDH activity is reduced in the BZ. For this, we assessed the upregulated pathways in the BZ using the already published RNA-seq data in the BZ and RZ (36). Analysis of these data revealed changes in a number of proteins, including the mRNA-binding protein TTP (FIG. 10A). We confirmed that TTP levels are increased in the BZ by measuring its mRNA and protein levels in the BZ and RZ (FIG. 3A-C).

Since TTP can regulate the expression of proteins by degrading their mRNAs, we then assessed whether TTP can regulate α-KGDH activity. For this, we generated cardiac-specific Ttp^{-/-} mice (cs-Ttp^{-/-}, FIG. 3D, E), and studied whether TTP regulates LIAS and Lipo-DLST by measuring their levels in the RZ and BZ of WT and cs-Ttp^{-/-} mice. mRNA levels of LIAS and DLST were reduced in the BZ of WT mice, but did not change in cs-Ttp^{-/-} hearts (FIG. 10B). Additionally, while LIAS protein and Lipo-DLST levels were reduced in the BZ of wild type (WT) mice (FIG. 2F, G), this effect was significantly attenuated in cs-Ttp^{-/-} mice (FIG. 3F-H and FIG. 10C-E). Similar to WT hearts, the levels of OGDH, DLST and DLD proteins were not changed in the BZ of cs-Ttp^{-/-} hearts (FIG. 10F, G).

We next studied the mechanism by which TTP regulates LIAS. We identified several AREs in the Lias 3-UTR that are highly evolutionarily conserved across species (FIG. 10H,I), suggesting that TTP may regulate Lias mRNA through its binding and degradation. To confirm this, we performed an RNA co-immunoprecipitation (IP) experiment using anti-TTP or -control IgG antibody. We observed enrichment in a known TTP target (Tnf-α) in the TTP antibody group, but no enrichment for the negative control (hypoxanthine phosphoribosyltransferase 1; Hprt1). Lias mRNA levels were significantly enriched with TTP antibody compared with IgG control, indicating that Lias mRNA interacts with TTP protein (FIG. 3I). Additionally, RNA stability assays showed that overexpression (OE) of WT TTP is associated with a significant reduction in mRNA stability of Lias after treatment with actinomycin D (AcD) (FIG. 3J).

We also assessed the effects of TTP OE and showed that OE of WT TTP as opposed to C124R zinc finger TTP mutant (i.e., mutation in the TZF, rendering TTP unable to bind to AREs) in H9c2 cardiomyoblast cell line results in a significant decrease in LIAS and Lipo-DLST protein levels (FIG. 3K-M) and α-KGDH activity (FIG. 3N). Together, these results indicate that TTP causes a decrease in the stability of Lias mRNAs and a reduction in their protein levels in addition to a reduction in the lipoylation of DLST, likely due to the reduced LIAS levels.

LIAS can lipoylated several proteins, including pyruvate dehydrogenase (PDH). Given PDH's role in the conversion of pyruvate into acetyl-coA, we next assessed whether TTP regulation may affect PDH activity and the change we have observed in the TCA metabolites. We first showed that unlike Lipo-DLST, the lipoylation of Dihydrolipoyl transacetylase (DLAT, the E2 component of PDH that is lipoylated by LIAS) is not reduced in the BZ of WT or cs-Ttp^{-/-} mice after MI or with TTP OE (FIG. 10J, K). Additionally, our tracing studies indicated that there is in fact reduced labeling of pyruvate and no change in lactate in the BZ (FIG. 3O, P). Additionally, labeled citrate: labeled pyruvate was not changed in the BZ (FIG. 10L, M), arguing against a reduction in PDH activity in the BZ. Finally, we measured PDH activity in the RZ and BZ of WT and cs-Ttp^{-/-} heart and showed that there was no difference in their activity (FIG. 3Q, R). These results indicate that the regulation of LIAS by TTP is sufficient to change the lipoylation of DLST with the resultant reduction of α-KGDH activity, but this regulation is not sufficient to regulate the PDH activity in the heart, highlighting the differential regulation of LIAS targets through its regulation by TTP. TTP binds to mRNA and degrades it; TTP degrades LIAS to decrease lipoylation of DLST, which reduces α-KGDH activity.

Deletion of Ttp Reverses the Metabolic Changes of the BZ after MI

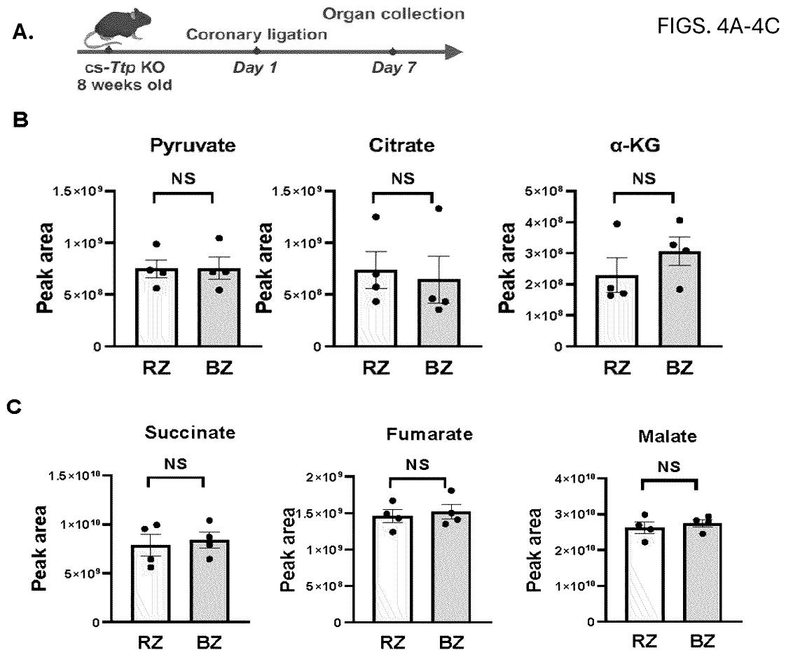

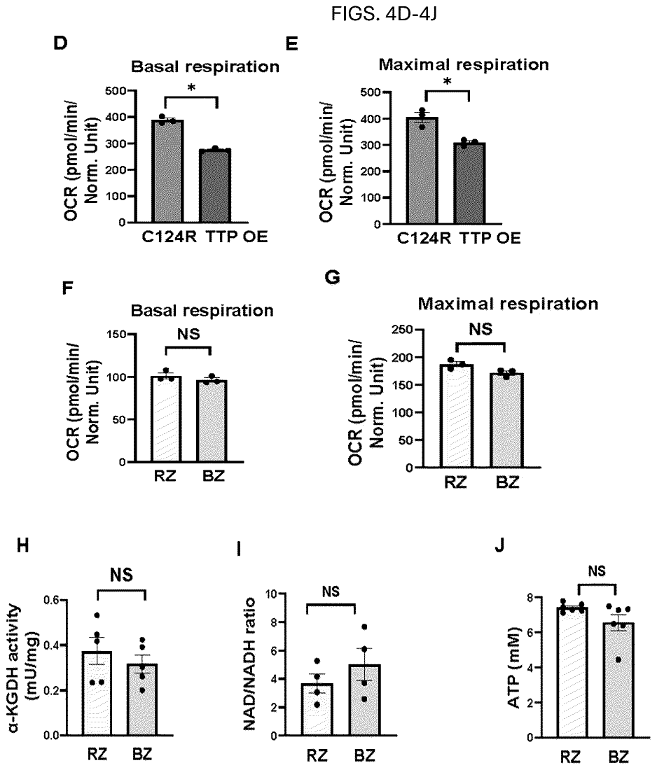

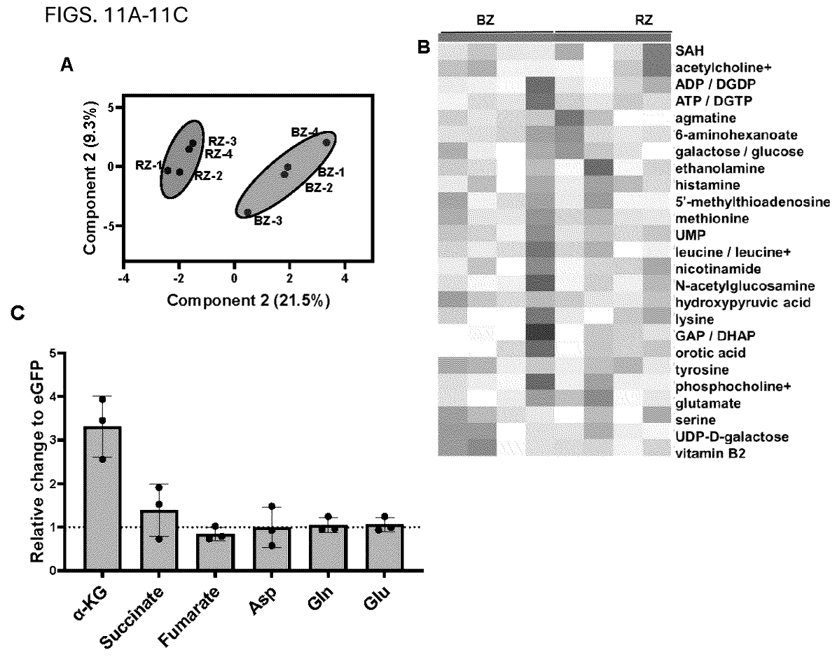

We next assessed whether Ttp deletion would result in the reversal of the metabolomic changes that are noted in the BZ after MI. To assess this, we subjected cs-Ttp^{-/-} mice to MI operation and performed metabolomics in the RZ and BZ (FIG. 4A). PC analysis showed that the BZ and RZ samples also cluster separately in cs-Ttp^{-/-} hearts (FIG. 11A). The metabolic block at the α-KGDH step noted in WT mice was not observed in the BZ of cs-Ttp^{-/-} hearts, as the levels of metabolites upstream of this step (i.e., pyruvate, citrate and α-KG) are not increased (FIG. 4B and FIG. 11B). TCA metabolites downstream from α-KGDH were also not changed in the BZ of cs-Ttp^{-/-} hearts compared to the RZ (FIG. 4C). These results support that TTP mediates the blockage at the level of α-KGDH. We then assessed whether TTP regulates mitochondrial respiration using the Seahorse system. OE of TTP in H9c2 cells resulted in a decrease in the basal and maximal respiration (FIG. 4D, E), confirming that TTP regulates the TCA cycle and mitochondrial respiratory function. We also measured OCR in the BZ of WT and cs-Ttp−/− mice and showed that while basal and maximal respiration is reduced in the BZ of WT mice compared to RZ (FIG. 1G, H), there is no difference in cs-Ttp^{-/-} mice (FIG. 4F,G). Additionally, α-KGDH activity was not different between the BZ and RZ in the cs-Ttp^{-/-} hearts (FIG. 4H), while it was lower in the BZ (FIG. 1F) and with TTP overexpression (FIG. 3N). There was also no difference in NAD/NADH and ATP levels or α-KGDH activity in the BZ vs RZ of cs-Ttp^{-/-} hearts (FIG. 4I, J), although they were different in the WT hearts (FIG. 1H, I). Finally, we performed metabolomics on H9c2 cells overexpressing TTP or control and assessed the metabolites in the TCA cycle. These results again demonstrated an increase in α-KG levels (FIG. 11C), supporting that an increase in TTP results in a block at the levels of α-KGDH. These results further support a role for TTP in metabolic changes in the BZ.

### TTP Deletion Protects Against Ischemic Injury Through α-KGDH

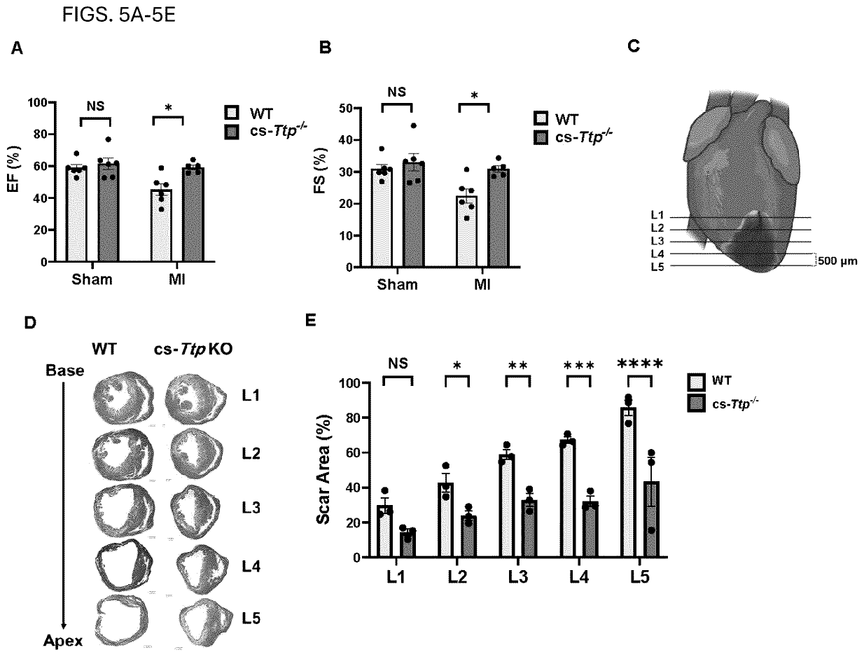

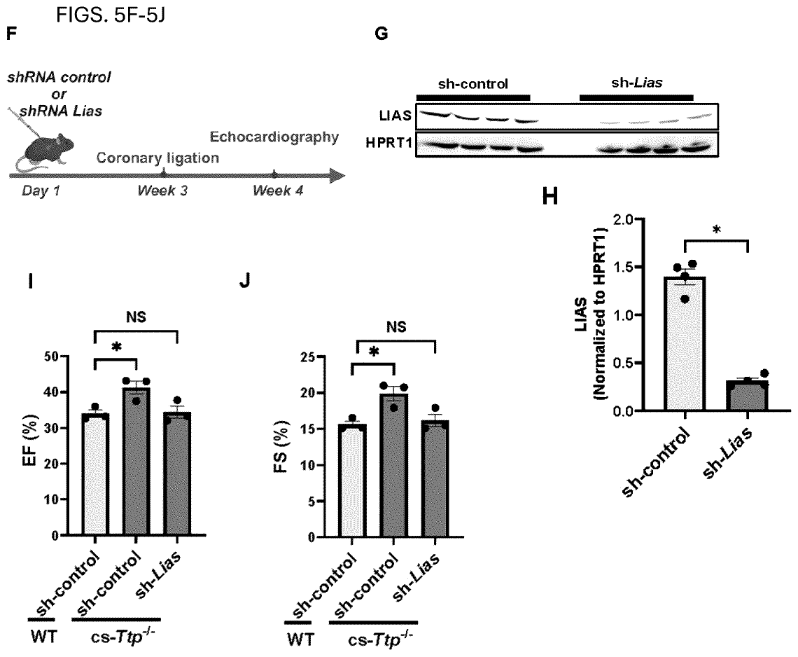

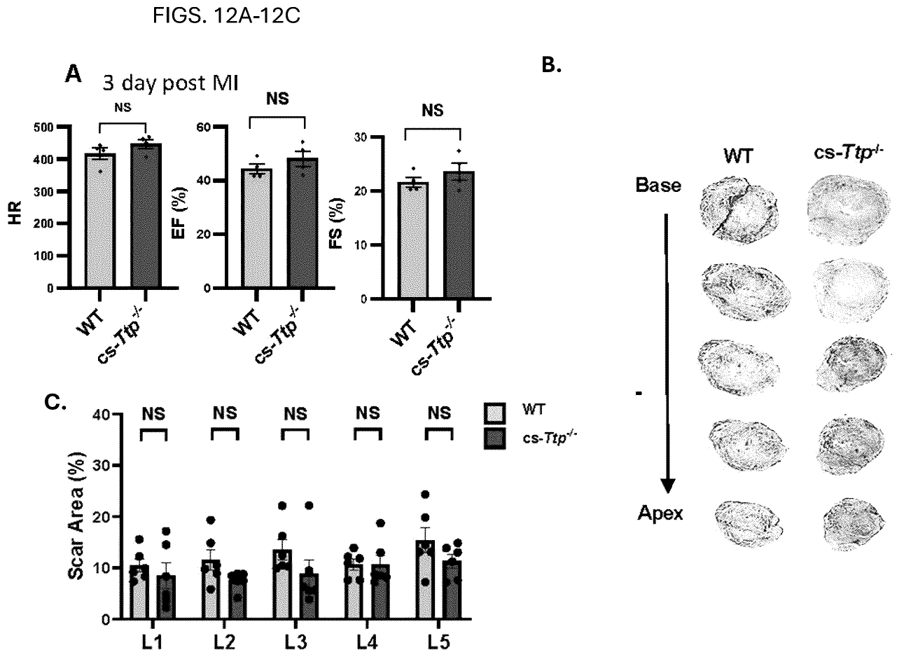

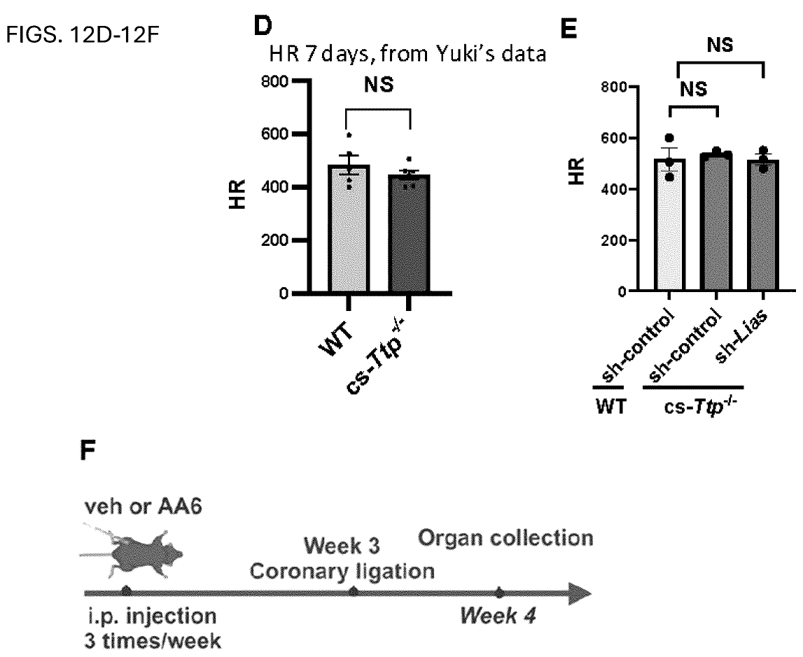

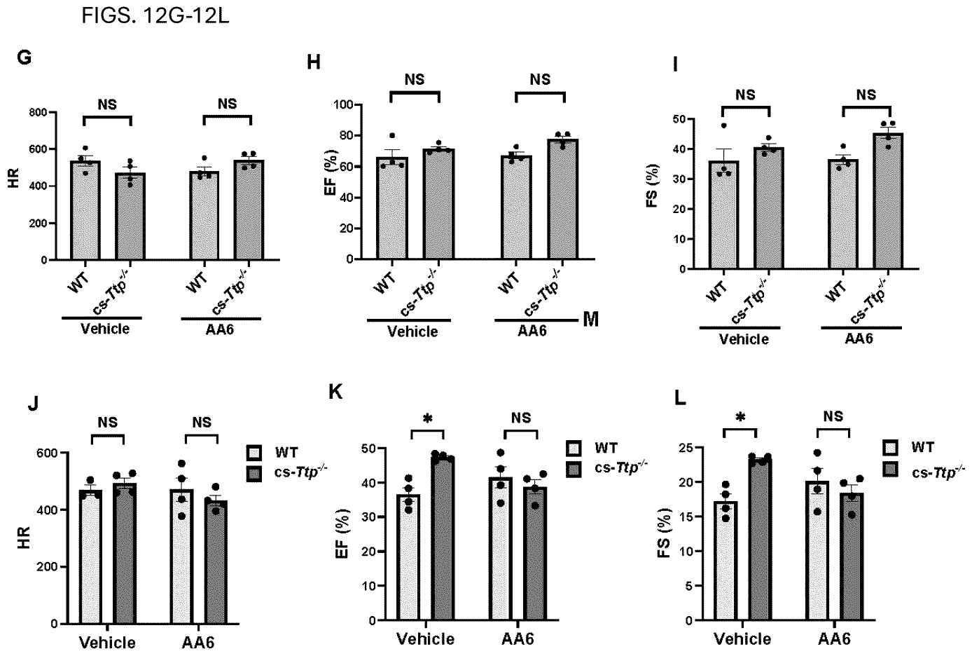

Since TTP inhibits the activity of α-KGDH and mitochondrial activity, we then tested whether deletion of Ttp would protect against ischemic damage in the heart. To assess this, we subjected WT and cs-Ttp^{-/-} mice to MI and assessed cardiac function using echocardiography 7 days after MI. we first showed that 3 days after MI, cardiac function and cardiac damage is not different between WT and cs-Ttp^{-/-} mice (FIG. 12A-C), indicating that coronary ligation results in the same degree of damage in these mice. To determine whether the repair mechanism is different between the WT and cs-Ttp^{-/-} mice, we assessed cardiac function 7 days after MI. Our results showed that cs-Ttp^{-/-} mice display improved cardiac function, as assessed by ejection fraction (EF) and fractional shortening (FS) (FIG. 5A, B and FIG. 12D). We also assessed the degree of damage to the heart with MT staining, which also demonstrated less damage in the cs-Ttp^{-/-} hearts (FIG. 5C-E). These results indicate that TTP has deleterious effects in the heart against MI, as its deletion has beneficial effects during the remodeling process, while the degree of damage during the ischemic period (i.e., 3 days after MI) is not altered with Ttp deletion.

We next performed experiments to assess whether the protective effects of Ttp deletion is through the effects of TTP on α-KGDH. For this, we took two approaches: 1) we injected an adeno-associated virus (AAV) of Lias shRNA into the heart, and 2) we used a specific chemical inhibitor of α-KGDH, (S)-2-[(2,6-dichlorobenzoyl) amino] succinic acid (AA6).

For the Lias shRNA AAV, we injected the hearts of WT and cs-Ttp^{-/-} mice with 1×10⁸ plaque-forming unit (PFU) of the AAV, followed by the induction of MI 3 weeks later and assessment of cardiac function after 7 days, as described previously (37) (FIG. 5F). We first showed that treatment with Lias shRNA AAV results in a significant decrease in LIAS protein in the heart (FIG. 5G, H). Our results indicated that the protective effects of Ttp deletion are reversed with LIAS knockdown, along with a significant decrease in EF and FS (FIG. 51, J and FIG. 12E).

For AA6 treatment, we treated WT and cs-Ttp^{-/-} mice with intraperitoneal (IP) injection of the chemical 3 times a week for 3 weeks, followed by the induction of MI and assessment of cardiac function 1 week later (FIG. 12F). First, we showed that sham operation does not result in a change in heart rate (HR) and cardiac function in the presence or absence of AA6 treatment (FIG. 12G-I). Treatment with AA6 resulted in no change in HR (FIG. 12J), but led to a reversal in the protection of the cs-Ttp^{-/-} mice against injury 7 days after MI, as assessed by measurement of EF and FS (FIG. 5K,L). Overall, these results indicate that TTP plays a critical role in cardiac repair and recovery after MI through its regulation of α-KGDH and the TCA cycle.

### Infiltration of the Inflammatory Cells in the BZ Induces TTP Expression

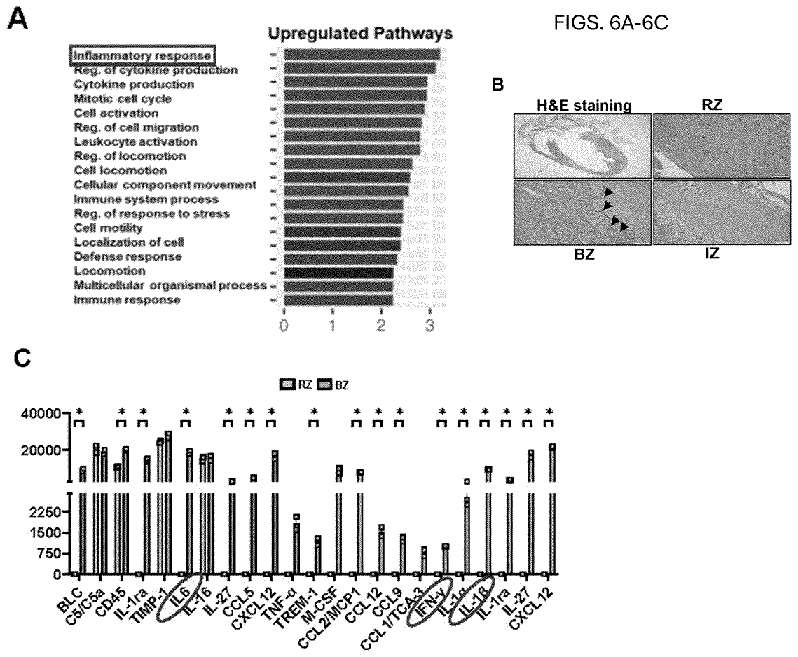

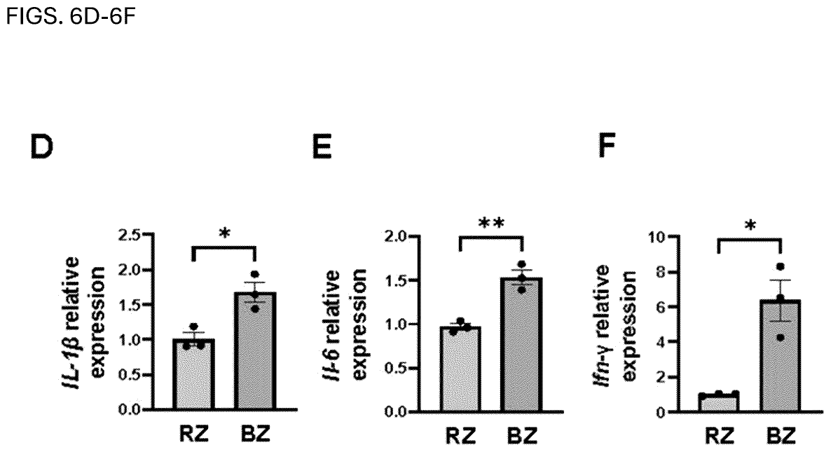

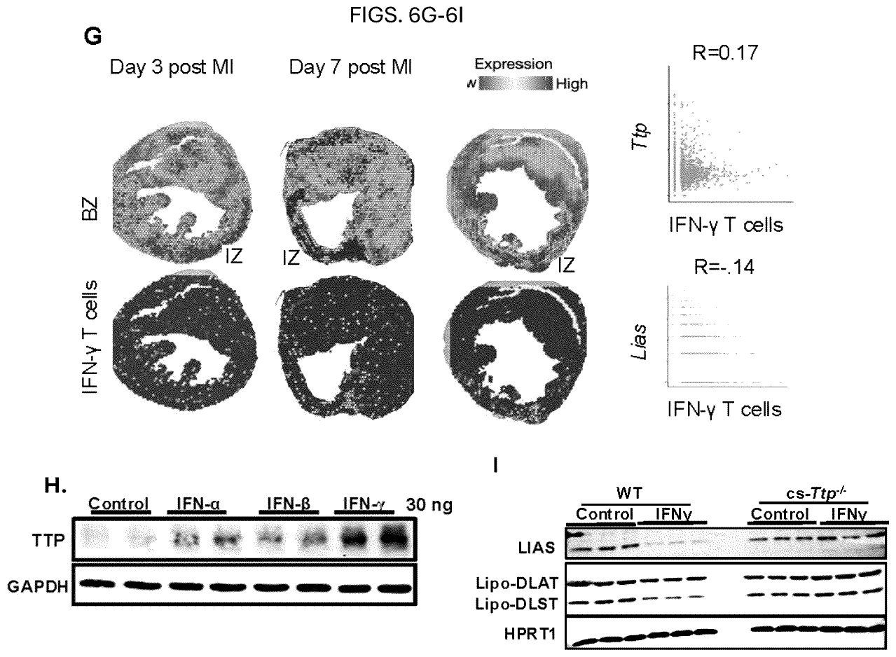

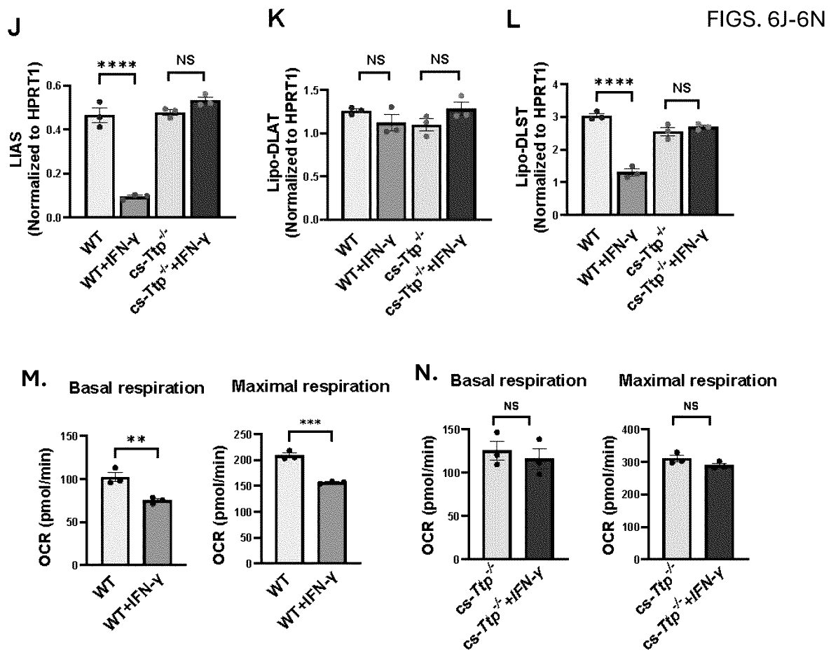

We next assessed the mechanism for the TTP-mediated changes in CM metabolic changed. Since TTP is induced by inflammatory cytokines, we posited that TTP induction in the BZ CMs is likely through the effects of inflammatory cytokines in that region. We first demonstrated that the inflammatory response is the most upregulated pathway in the BZ compared to the RZ using RNA-seq data from the BZ and RZ (36) (FIG. 6A). H&E staining confirmed that there is significant infiltration of the inflammatory cells in the BZ (FIG. 6B).

To investigate the role of inflammatory response in the BZ, we first performed Gene Set Enrichment Analysis (GSEA) on publicly available RNA-seq data derived from multi-omic studies of myocardial infarction (4). The GSEA analysis identified several statistically significant pathways enriched in the BZ after MI. The top enriched pathways include Interferon Gamma Response and Interferon Alpha Response, both are associated with immune and inflammatory responses, suggesting that interferons play an important role in the BZ immune response.

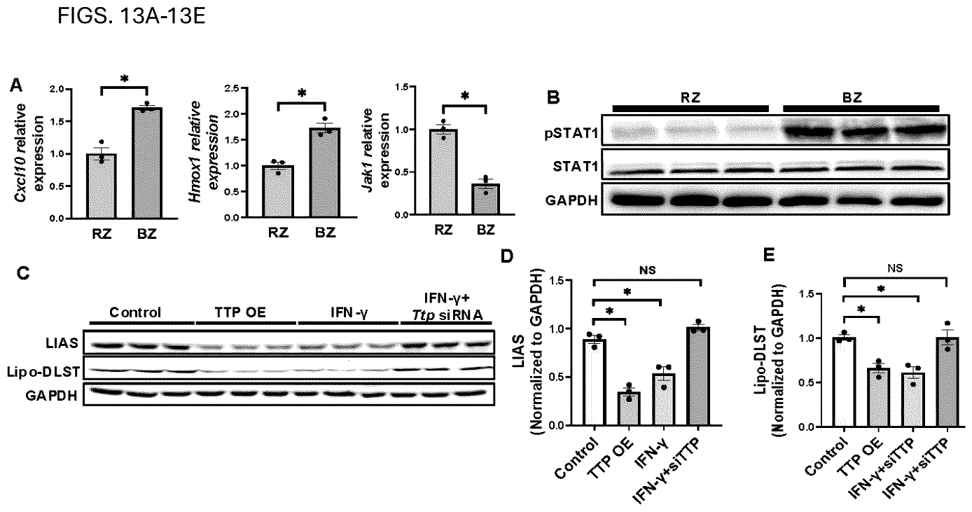

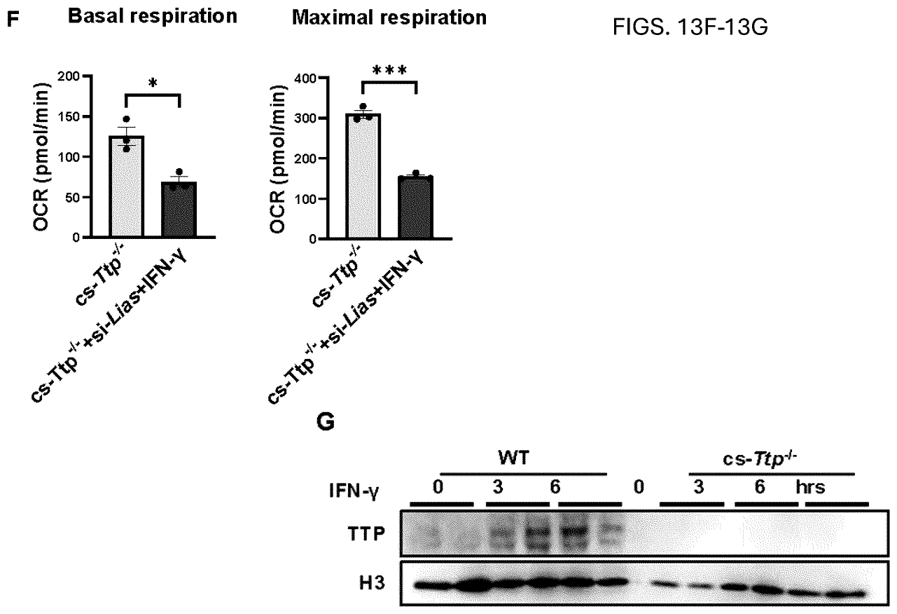

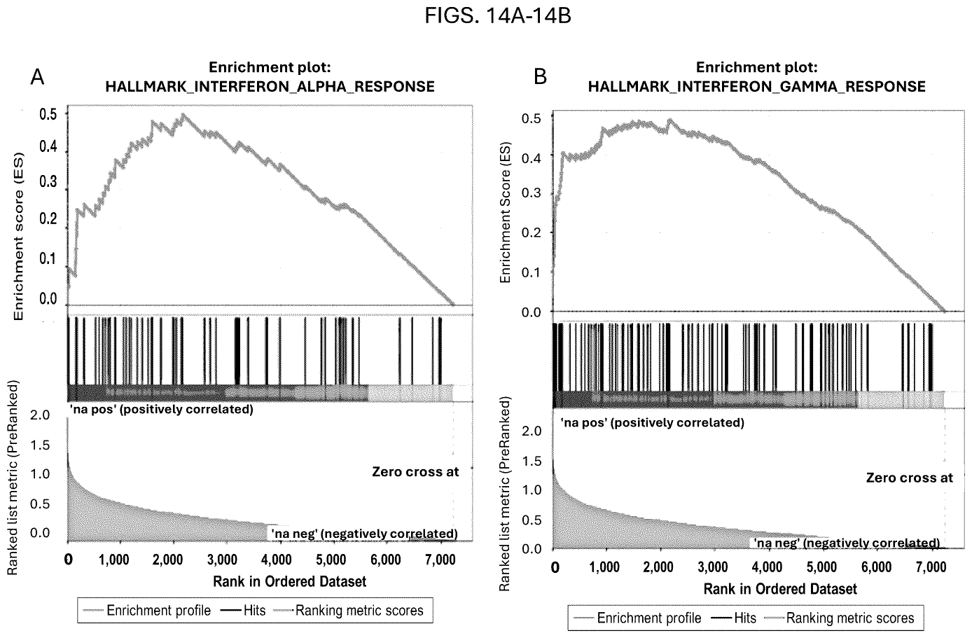

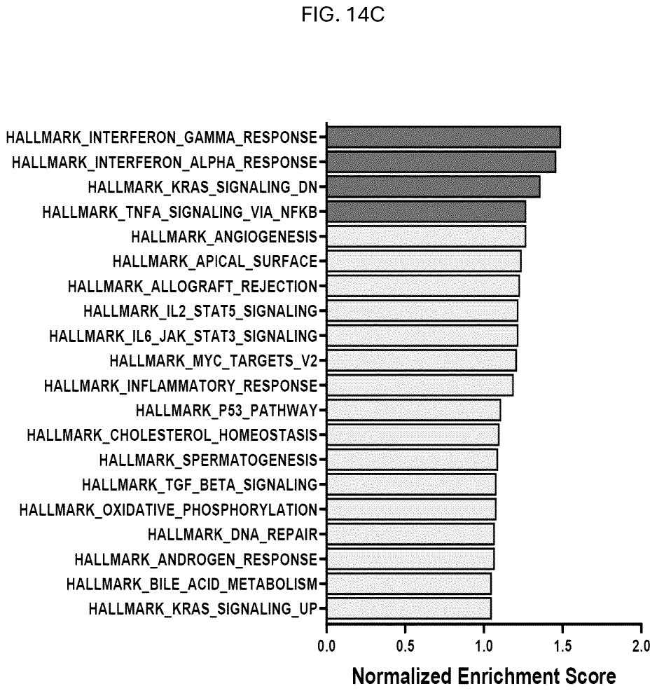

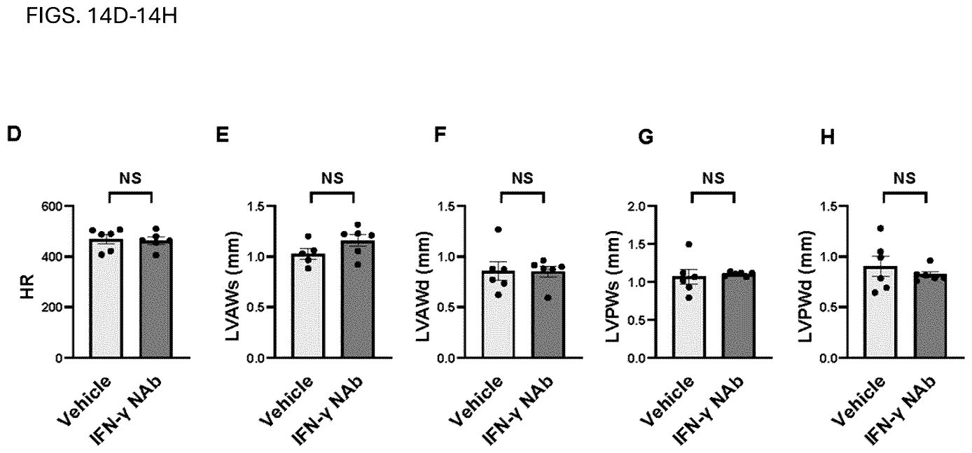

To confirm these results, we then assessed the levels of different cytokines in the BZ after MI, and demonstrated that a number of cytokines are increased in the BZ (FIG. 6C), highlighting the inflammatory nature of the BZ after MI. mRNA analysis of various cytokines also showed that the BZ has a higher level of a number of cytokines, including interleukin (IL)-1β, IL-6 and interferon-γ (IFN-γ) (FIG. 6D-F). Additionally, there was an increase in Cxcl10, Hmox1, and Jak1 mRNA and STAT1 phosphorylation in CMs of the BZ (FIG. 13A,B), indicative of increased intracellular signaling associated with IFN-γ (38). Overall, our data demonstrate increased levels of various cytokines in the BZ. However, since Type II IFN (i.e., IFN-γ) is relatively understudied in myocardial injury, we chose to further explore its contribution to the immune response in the BZ.

We then assessed whether these cytokines are associated with an increase in TTP levels and showed that treatment of isolated adult CMs with IFN-γ was associated with increased TTP mRNA and protein levels in a time-dependent manner, with TTP rising to its peak around 3-6 hours after treatment (FIG. 6G). To determine whether inflammatory cytokines alter the levels of the components of α-KGDH, we treated adult CMs with INF-7 and showed a significant decrease in LIAS and Lipo-DLST in isolated adult CMs from WT hearts, but not from cs-Ttp^{-/-} with IFN-γ (FIG. 6H-J). We also measured the levels of LIAS and Lipo-DLST in H9c2 cells treated with control, TTP OE, IFN-γ, and with IFN-γ and Ttp knockdown (KD). These data demonstrated that while the levels of these proteins are reduced with both TTP OE and IFN-γ treatment, Ttp KD reversed the effects of IFN-γ and caused a relative increase in their levels (FIG. 13C-E), indicating that cytokines regulate LIAS and subsequent lipoylation of DLST through TTP. Finally, we measured OCR in isolated CMs from WT and cs-Ttp^{-/-} hearts in the presence and absence of IFN-γ and showed that both basal and maximal respiration are decreased with IFN-γ (FIG. 6M). These changes were not noted in isolated CMs from cs-Ttp^{-/-} mice (FIG. 6N), however, knockdown of Lias using an siRNA led to a decrease in basal and maximal respiration in cs-Ttp^{-/-} CMs treated with IFN-γ (FIG. 13F), indicating that LIAS inhibition in cs-Ttp^{-/-} hearts can mimic the effects of cytokines on OCR found in WT CMs. Together, these results indicate that ischemic damage is associated with the infiltration of the BZ by the inflammatory cells, which results in increased production of TTP in CMs in the BZ.

TABLE 2
Overlap of mRNA-binding protein targets
with downregulated genes in myocardial infarction

| mRNA | | Total Target | | | |
|---|---|---|---|---|---|
| Binding | Downregulated | Genes in | Percent | | |
| Protein | Target Count | File | Overlap | log2FoldChange | pvalue |
| AGO2 | 2011 | 9244 | 44.5898 | −0.18792 | 0.444095 |
| APC | 2376 | 11199 | 52.68293 | 0.685681 | 0.026767 |
| CELF1 | 709 | 2174 | 15.72062 | −0.01176 | 0.969143 |
| CELF2 | 1312 | 4844 | 29.09091 | −0.28794 | 0.346727 |
| CELF4 | 1887 | 7282 | 41.84035 | 1.088694 | 0.034132 |
| CIRBP | 1192 | 4690 | 26.43016 | 0.585945 | 0.139742 |
| CPSF6 | 1077 | 3968 | 23.88027 | 0.097597 | 0.751973 |
| CREBBP | 432 | 2171 | 9.578714 | 0.914547 | 0.009082 |
| ELAVLI | 223 | 763 | 4.944568 | −0.64694 | 0.081027 |
| EZH2 | 2304 | 10271 | 51.08647 | −1.08278 | 0.022101 |
| FAM120A | 987 | 4067 | 21.8847 | 0.175857 | 0.401022 |
| FMR1 | 305 | 1141 | 6.762749 | −0.11453 | 0.751371 |
| FUS | 2211 | 9458 | 49.02439 | −0.23648 | 0.250773 |
| HNRNPR | 2499 | 10962 | 55.4102 | −0.36643 | 0.130415 |
| MBNL1 | 1009 | 3614 | 22.37251 | −0.14267 | 0.574586 |
| MBNL2 | 503 | 1668 | 11.15299 | −0.03397 | 0.871773 |
| MSI2 | 1092 | 4478 | 24.21286 | −0.15948 | 0.554026 |
| NOVA1 | 617 | 1948 | 13.68071 | 0.326098 | 0.423553 |
| NOVA2 | 915 | 2534 | 20.28825 | −0.04376 | 0.941764 |
| PABPC1 | 938 | 4933 | 20.79823 | −0.26998 | 0.467582 |
| PSRSF3 | 2376 | 11199 | 52.68293 | NA | NA |
| PTBP1 | 546 | 1433 | 12.10643 | 0.936411 | 0.0111 |
| PTBP2 | 1142 | 4580 | 25.32151 | −0.37333 | 0.406932 |
| RBFOX2 | 2378 | 11255 | 52.72727 | −0.74608 | 0.066658 |
| RBFOX2 | 2378 | 11255 | 52.72727 | 1.054945 | 0.004203 |
| SRCF2 | 617 | 1948 | 13.68071 | NA | NA |
| SRRM4 | 1560 | 8312 | 34.5898 | 0.077943 | 0.837173 |
| SRSF1 | 1032 | 4633 | 22.88248 | −1.07888 | 0.006015 |
| SRSF2 | 1296 | 6032 | 28.73614 | 0.425579 | 0.294602 |
| SRSF4 | 418 | 2391 | 9.268293 | −0.49903 | 0.370964 |
| SRSF7 | 2111 | 8878 | 46.8071 | −0.19035 | 0.552487 |
| TAF15 | 758 | 2322 | 16.8071 | 1.094404 | 0.014096 |
| TARDBP | 2147 | 8520 | 47.60532 | −0.72609 | 0.110163 |
| U2AF2 | 1923 | 8543 | 42.63858 | 1.297273 | 0.001906 |
| UPF1 | 266 | 1081 | 5.898004 | 0.926675 | 0.002211 |
| YTHDC2 | 2353 | 13329 | 52.17295 | −0.31948 | 0.434663 |
| YY1 | 921 | 4250 | 20.42129 | 1.306374 | 0.008262 |
| TTP | 918 | 3742 | 20.35477 | 1.67307 | 0.001242 |

### Inhibition of IFN-γ Rescues Metabolic Alterations in the BZ and Protects Against Injury

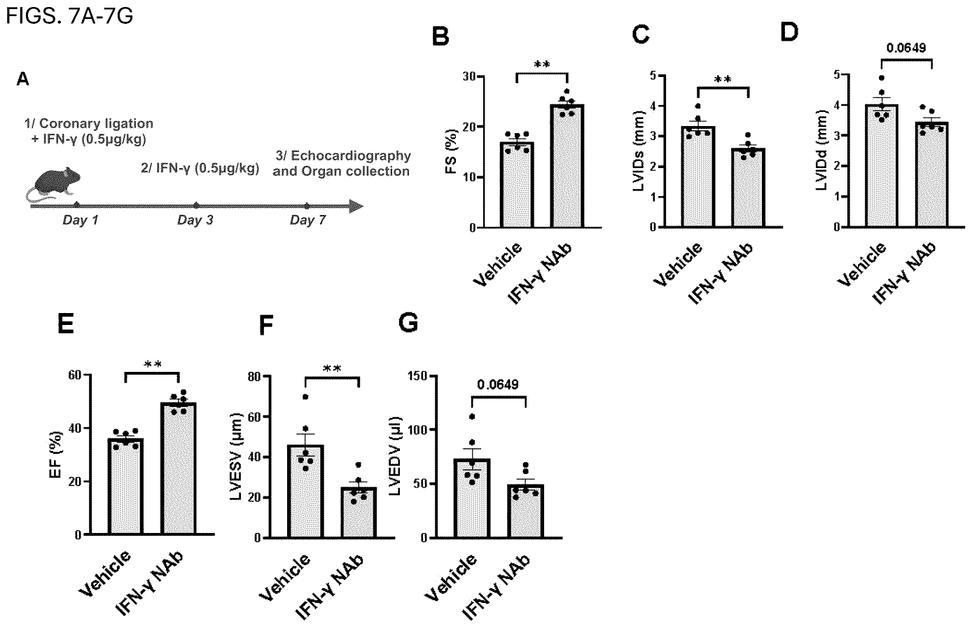

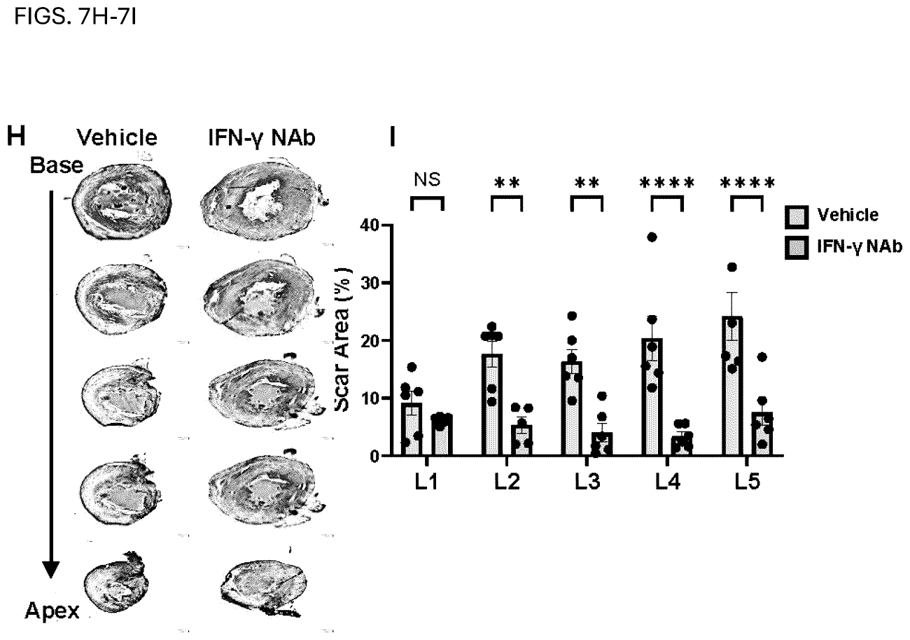

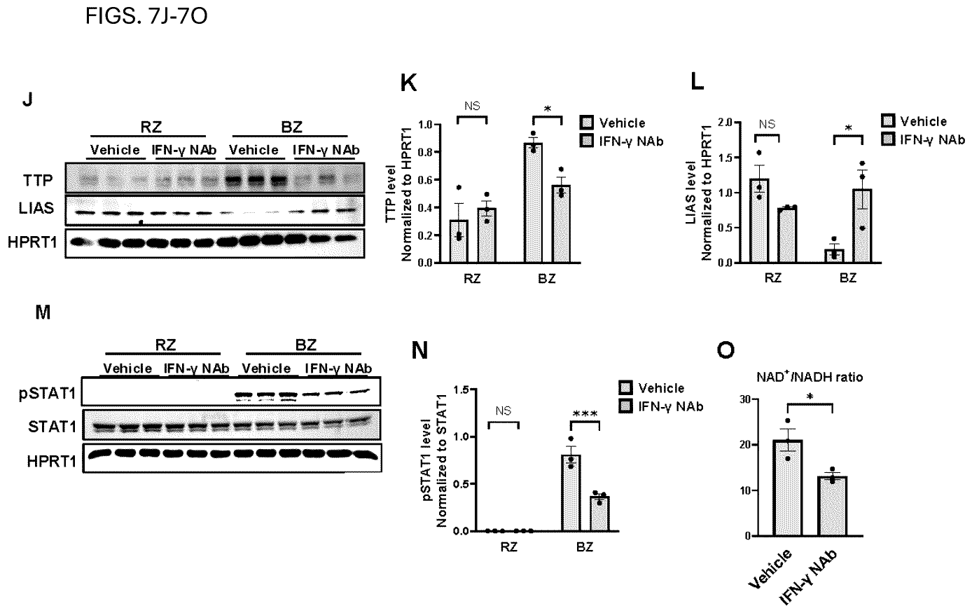

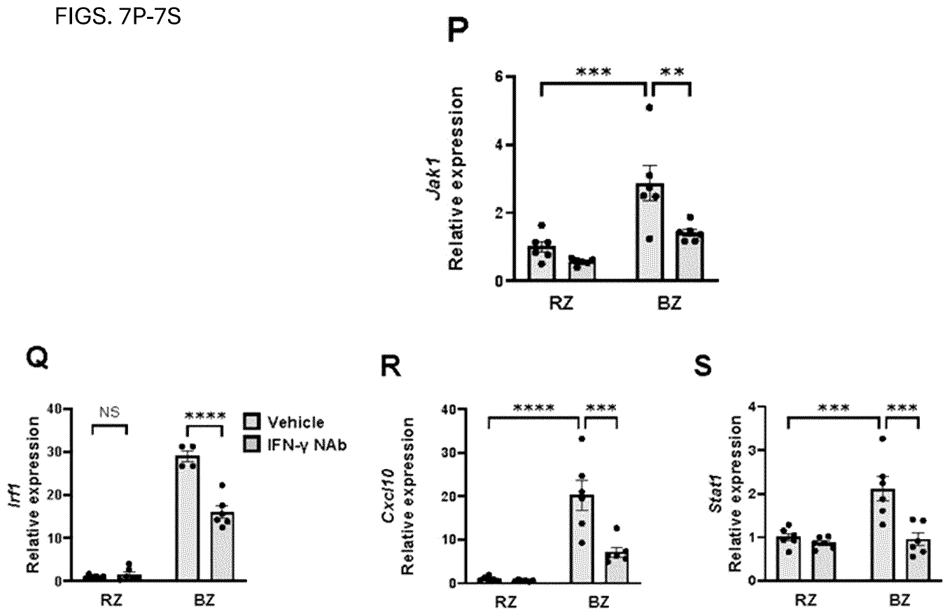

Our data indicate that IFN-γ and IL-6 both cause an increase in TTP levels in BZ CMs, which in turn, leads to an inhibition of ax-KGDH activity and worsened cardiac function. We next studied whether targeting these cytokines can improve cardiac function after MI. We specifically focused on IFN-γ since little is known about type 2 interferons in cardiac remodeling after MI. We then treated mice via intraperitoneal (IP) injection with 50 ng/kg of IFN-γ neutralizing antibody on the day of coronary ligation and day 3 post-MI (FIG. 7A). Cardiac function, as assessed by echocardiography on day 7, showed reduced chamber dilation and improved contractile function in the IFN-γ neutralized group (FIG. 7B-G), indicating that IFN-γ antibody results in improved cardiac function and cardiac structure after MI. Consistent with these findings, MT staining showed less cardiac damage and fibrosis in the IFN-γ neutralizing antibody-treated group compared to the vehicle-treated group (FIG. 7H, I). These results support that inhibition of IFN-γ signaling has beneficial effects on the heart after MI, highlighting the role of this cytokine in cardiac recovery. We next assessed whether inhibition of IFN-γ signaling has an effect on the expression of key proteins that we identified to be altered in the BZ. Western blot analysis revealed that TTP levels, which were elevated in the BZ of the vehicle-treated group, were significantly reduced in the group treated with the IFN-γ neutralizing antibody. Moreover, LIAS expression and Lipo-DLST, which were decreased in the BZ in vehicle-treated mice, were rescued by IFN-γ antibody treatment (FIG. 7J-L). pSTAT1 levels were reversed by IFN-γ antibody (FIG. 7M and N). qPCR analysis of Stat1, Jak1, Irf1 and Cxcl10 confirmed the activation of the IFN-γ signaling pathway in the BZ after MI and IFN-γ antibody treatment reversed the upregulation of these genes (FIG. 7N-Q), suggesting effective inhibition of IFN-γ signaling in the BZ 7 days post-MI.

### Discussion

Remodeling is an important step in cardiac recovery after MI. A number of processes have been proposed to play a role in this remodeling, including infiltration of immune cells and the development of fibrotic tissue by fibroblasts (9). The BZ is a key region for cardiac remodeling after MI (2, 3, 5, 8), however, the mechanism by which CMs in the BZ are regulated after MI are not well known. In this paper, we provide a functional link between immune cells and CMs and demonstrate that immune cells have detrimental effects on CMs by inhibiting the TCA cycle at the level of α-KGDH. This will result in a reduction in OCR by the mitochondria at a time when CMs likely need higher energy to compensate for the damage in the infarcted region. Overall, the activation of this pathway leads to detrimental effects in the heart and targeting this pathway by inhibiting TTP may have potential therapeutic effects after MI.

Our results showed that treatment with IFN-γ neutralizing antibody at day 1 and 3 post MI significantly improves cardiac function, and reduces ventricular dilation and the damaged area in the mouse heart. By neutralizing IFN-γ, we observed a preservation of α-KGDH activity and oxygen consumption rates. Moreover, IFN-γ neutralization resulted in decreased TTP production in BZ and reversed the reduced lipoylation of DLST in the BZ. This suggests that IFN-γ plays a key role in driving metabolic dysfunction in the BZ, contributing to adverse remodeling and decreased cardiac function post-MI. In the heart, IFN-γ is secreted by the infiltrated natural killer cells, CD8+ T cells, CD4+T helper type 1 cells, dendritic cells, and a subtype of macrophages (40). IFN-γ levels are elevated in patients with acute myocardial infarction and correlates with the expansion of pro-inflammatory myeloid cells (41). While recent studies have demonstrated that type I interferon exacerbate cardiac dysfunction and inflammation post-MI (42-45), our study extends these observations to type II interferons, which drive chronic inflammation and adverse cardiac remodeling. By administering an IFN-γ neutralizing antibody post-MI, we observed a significant improvement in cardiac function, accompanied by reduced damage in the BZ, highlighting that IFN-γ, like type I interferons, plays a critical role in fueling the inflammatory cascade that leads to metabolic impairment and adverse remodeling in the BZ.

In summary, our findings provide novel insights into the role of IFN-γ in post-MI cardiac function, specifically in the BZ. We demonstrate that neutralizing IFN-γ preserves mitochondrial metabolism (α-KGDH activity and oxygen consumption) and improves cardiac function, highlighting a new perspective on cytokine signaling in post-MI metabolism. The mechanistic link between IFN-γ, TTP, and α-KGDH inhibition in CMs further deepens our understanding of IFN-γ's impact on cardiac remodeling. This study suggests that targeting IFN-γ could offer a promising therapeutic approach for post-MI treatment, distinct from traditional strategies focused on general inflammation.

### Materials and Methods

### Mouse Studies

All studies adhered to the National Institutes of Health Guide for the Care and Use of Laboratory Animals; the protocols were approved by Institutional Animal Care and Use Committee of Northwestern University.

Animals were housed with 12 h dark and light cycles with free access to traditional chow and water. Both sexes, 8-12-week-old adults were used for experiments. Before harvesting hearts or other tissue, mice were deeply anaesthetized with freshly prepared avertin and killed by cervical dislocation.

WT C57BL/6 mice were purchased from Taconic. C57BL/6 Ttpfl/fl mice (a gift from Perry Blackshear, National Institute of Environmental Health Sciences [NIEHS]) have flox sites flanking exon 2 of TTP. cs-Ttp−/− mice were generated by breeding Ttpfl/fl mice with αMHC-Cre transgenic mice (Jackson labs). Ttp cardiomyocyte-specific deletion due to Cre-recombinase expression in hepatocytes was confirmed at the mRNA and protein levels. Both male and female mice were used in the studies.

AAV constructs containing shRNA against Lias were packaged into AAV serotype 9 viral particles. Five-week-old mice were subjected to intramyocardial injections of 1×10⁸ PFU of AAV9 containing control shRNA or shRNA targeting Lias. Under sterile conditions, a small incision was made along the line connecting the xiphoid and axilla, followed by blunt dissection of the pectoral muscles to expose the intercostal space. The heart was gently externalized, and a total of 30 μL of the adenovirus solution was injected into three sites (ventral, dorsal, and lateral wall) of the left ventricle using a Hamilton syringe with a 30 G needle. After injection, the heart was immediately returned to the thoracic cavity, and the incision was closed using a horizontal mattress suture with 5-0 silk. Three weeks post-injection, the mice were subjected to LAD (left anterior descending artery) ligation to induce myocardial infarction. Post-operative pain was managed with buprenorphine, and mice were monitored until full recovery. Cardiac function and expression of the target genes were assessed at designated time points post-ligation (47).

IFN-γ neutralizing antibody. Reactive Species: Mouse. Host Species: Rat. Immunogen: E. coli-Derived Recombinant Mouse IFN-γ. Endotoxin Level: <0.1 EU/μg (determined by the LAL method). Formulation: Lyophilized from modified Dulbecco's phosphate-buffered saline (1×PBS), pH 7.2-7.3; Contains 5.0% w/v trehalose; No calcium, magnesium, or preservatives; Filtered through a 0.2 μm filter.

### Cardiomyocyte Isolation

Cardiomyocytes were isolated from WT C57/B6J and cs-Ttp−/− mice aged 8 to 12 weeks following standard protocols. Mice were anesthetized, and the chest was opened to expose the heart. After cutting the descending aorta, the heart was flushed with 7 mL of EDTA buffer through the right ventricle, clamped at the ascending aorta, and transferred to a dish with fresh EDTA buffer. The heart was then digested by sequentially injecting 10 mL of EDTA buffer, 3 mL of perfusion buffer, and 30 to 50 mL of collagenase buffer into the left ventricle. The heart chambers were separated and gently torn into 1-mm pieces, followed by gentle trituration to complete cellular dissociation, with enzyme activity halted using 5 mL of stop buffer. The resulting cell suspension was filtered through a 100-μm filter and subjected to 4 rounds of gravity settling with intermediate calcium reintroduction buffers to restore physiological calcium levels. The enriched cardiomyocyte pellet from each round was used for subsequent experiments, while the supernatants were combined to isolate nonmyocyte cardiac populations. Cardiomyocyte viability and yield were assessed using a hemocytometer. The isolated cardiomyocytes were then plated on laminin-coated tissue culture plastic or glass coverslips in a humidified incubator (37° C., 5% CO2), with media changes every 48 hours.

### Echocardiography, Organ Harvest, and Histological Analysis

Mice were anesthetized with isoflurane administered via a nasal cone, and their chests were shaved. Their body temperature was kept at 37° C., and heart rates were continuously monitored. Transthoracic echocardiography was conducted using the VisualSonics Vevo 2100 system with an MS400 transducer (FUJIFILM). Left ventricular ejection fraction (LVEF), along with other systolic function indices and LV wall thickness, were measured from short-axis M-mode scans at the midventricular level, identified by the presence of papillary muscles. Throughout the echocardiogram, isoflurane was kept at 1.0-1.5% to maintain a heart rate between 400-450 beats per minute. All measurements were taken at least three times, with the averages reported. At the time of tissue harvest, mice were anesthetized with 250 mg/kg dose of freshly prepared Tribromoethanol (Avertin). Tissue was excised, rinsed in PBS to remove excess blood, and was then freshly frozen in liquid nitrogen and stored at −80° C. For histological analysis, immediately after making a small incision in the inferior vena cava, beating heart was perfused with relaxing buffer (100 mM KCl, 5 mM EGTA, and 5 mM Na-pyrophosphate) until termination of beating, followed immediately with 30 ml of 4% paraformaldehyde using the peristaltic pump (7 mL/min) from the apex of left ventricle through a 22-gauge needle. The excised heart tissue was further fixed overnight in 4% paraformaldehyde before graded dehydration in 70%, 80%, 90%, and 100% ethanol. The tissue sample was further dehydrated with xylene and embedded
into paraffin. Sections were stained with hematoxylin and eosin for evaluation of general cardiac morphology and tissue organization. Masson's trichrome staining was used to visualize cardiac fibrosis.

### RNA Isolation, Reverse Transcription and Quantitative RT-PCR

RNA was isolated from cells or tissues using RNA-STAT60 (Teltest) followed by chloroform extraction and precipitation. Reverse transcription was carried out using qScript cDNA Synthesis Kit (Quanta Bio). The resulting cDNA was amplified quantitatively using PerfeCTa SYBR Green Mix (Quanta Bio) on a 7500 Fast Real-time PCR System (Applied Biosystems). The relative gene expression was determined using differences in Ct values between gene of interest and house-keeping control genes. Complete list of primers can be found in Key resources table.

mRNA Stability Assay and RNA Co-IP

Cells were treated with 7.5 μM actinomycin D (MilliporeSigma) for indicated times, and RNA was harvested and processed as described above. RNA Co-IP experiments were conducted as described previously (48).

### RNA Co-IP Experiments

RNA co-IP was performed as previously described (REF). Briefly, H9c2 cells were grown in complete media, collected, resuspended in Buffer A (10 mM Tris-HCl, pH 7.6, 1 mM KAc, 1.5 mM MgAc, 2 mM DTT [MilliporeSigma], 10 μl/ml ProteaseArrest inhibitors [G-Biosciences]), and lysed using Power Gen 500 homogenizer (Thermo Fisher Scientific), followed by centrifugation at 12,000 g for 10 minutes at 4° C. to remove debris. Protein G Sepharose Fast Flow beads (MilliporeSigma) were incubated with human TTP or IgG antibody in the IP Buffer (10 mM Tris-HCl, pH 7.6, 1.5 mM MgCl2, 100 mM NaCl, 0.5% Triton X-100 [MilliporeSigma], 10 μl/ml ProteaseArrest inhibitors) at 4° C. with continuous rotation for 4 hours. Beads were then washed 3-6 times with cold IP buffer, followed by incubation with lysate at 4° C. with continuous rotation for 2 hours, 6 washes with cold IP buffer to remove unbound RNA, and RNA collection as described above. Equal amounts of RNA were amplified by qPCR using 2 pairs of primers targeting Lias, as well as the primers designed to target the regions near the 3-UTR of known targets of TTP (TNF-α) and negative control genes (HPRT1). Expression of each gene was normalized to that of 18S or β-actin, and data were expressed as fold enrichment over IgG control.

Stable Overexpression of TTP and its Mutant from in H9c2 Cells

To overexpress WT TTP and mutant TTP (C124R), H9c2 cells after three passages in a 35 mm well were incubated with 25 μl of concentrated lentiviruses, 775 μl of DMEM, and 0.8 μl of polybrene (Millipore Sigma) for 8 hours. Following this, 1 ml of DMEM was added, and the cells were incubated for an additional 16 hours. The media was then replaced with fresh DMEM without lentivirus, and the cells were used for subsequent experiments 48 hours after the media change (72 hours after the initial lentivirus incubation).

To establish stably overexpressing WT TTP and mutant TTP (C124R) H9c2 cells, 0.6 g/ml of puromycin was added for 7 days to select cells that had successfully integrated the gene of interest into their genome. The successful selection of these cells was confirmed by immunoblot and immunofluorescence.

### Transfection

siRNAs were purchased from Dharmacon. siRNAs were transfected using Dharmafect 1 Transfection Reagent (Dharmacon). Plasmids were transfected using Lipofectamine 2000 reagent (Invitrogen), Lipofectamine 3000 (Invitrogen), or calcium phosphate transfection. Experiments were performed 24~48 hours after transfection.

### Immunohistochemistry

Tissue sections on a cover glass were fixed with 4% paraformaldehyde for 15 minutes. The cells were then washed with PBS, blocked with 5% goat serum and 0.1% Triton X-100 in PBS for 60 minutes and incubated overnight with LIAS or lipoic acid antibody in blocking buffer. The bound antibodies were labeled with an Alexa Fluor® 488 anti-rabbit secondary antibody or an Alexa Fluor® 594 anti-rabbit secondary antibody, and nuclei were stained with DAPI. The cover glass was mounted on a slide glass and was visualized using a fluorescent microscopy.

### Human Samples

Slides from the autopsies containing representative regions of myocardium containing infarct and histologically normal tissue near the infarct border were selected for re-cutting. Paraffin blocks corresponding to those sections were re-cut in 4 micrometer thick sections by the Northwestern Pathology Core Facility. One slide was stained with hematoxylin and eosin to reexamine the infarct boundaries after the recuts. The infarct, boundary, and normal tissue were traced on the slide by an experienced anatomic pathologist. These markings were then applied to the unstained sections subjected to RNA-seq.

### Western Blots

Cells and tissue were lysed in radio-immunoprecipitation assay (RIPA) buffer supplemented with 1× protease inhibitor (G-Bioscience). Protein concentration in samples was determined using the BCA Protein Quantification Kit (Pierce). Equal amounts of protein were loaded onto a tris-glycine polyacrylamide gel (Life Technologies) and transferred to a nitrocellulose membrane. After blocking with tris-buffered saline containing 0.05% Tween 20 (DOT Scientific Inc) and 5% BSA, the membrane was incubated with primary antibody against proteins of interest. The antibodies that we used are: Anti-LIAS Rabbit Polyclonal Antibody (Proteintech, 11577-1-AP), Anti-DLST Antibody (Abcam, ab177934), DLD Polyclonal Antibody (Proteintech, 16431-1-AP), OGDH Polyclonal Antibody (Proteintech, 15212-1-AP), Anti-Lipoic Acid Antibody (Abcam, ab58724), Anti-SDHB Rabbit Polyclonal Antibody (Proteintech, 10620-1-AP).

[¹³C₆]-Glucose Tracing and Steady-State Metabolomics

For in vivo [¹³C6]-glucose tracing, mice were subjected to LAD coronary artery ligation to induce MI. Seven days post-MI, the mice were administered 200 μL of [[¹³C₆]-glucose solution (2 g/kg bodyweight) via oral gavage. After 4 hours, heart tissues were harvested, and the RZ and BZ were dissected. Metabolites were extracted using an 80% methanol and 20% ultrapure water solution. The extracted metabolites were then analyzed by the Metabolomics Core Facility at the Robert H. Lurie Comprehensive Cancer Center of Northwestern University. The samples underwent High-Performance Liquid Chromatography coupled with High-Resolution Mass Spectrometry and Tandem Mass Spectrometry (HPLC-MS/MS). The analysis was performed on a Thermo Q-Exactive system with an electrospray ionization source, paired with an Ultimate3000 HPLC system equipped with an Xbridge Amide column (Waters; 4.6 mm×100 mm, 3.5 μm particle size). The mobile phase A consisted of 95% water, 5% acetonitrile, 20 mM ammonium hydroxide, and 20 mM ammonium acetate at pH 9.0, while mobile phase B was 100% acetonitrile. The gradient profile used was as follows: 0 min, 15% A; 2.5 min, 30% A; 7 min, 43% A; 16 min, 62% A; 16.1-18 min, 75% A; 18-25 min, 15% A, at a flow rate of 400 μL/min.

The electrospray ionization source was operated with a capillary temperature of 275° C., sheath gas flow at 45 arbitrary units, auxiliary gas flow at 5 arbitrary units, and a spray voltage of 4.0 kV. Mass spectrometry data were acquired in positive/negative polarity switching mode with an m/z scan range of 70-850. MS1 data were collected at a resolution of 70,000, with an automatic gain control (AGC) target of 1×10\hat{ }6 and a maximum injection time of 200 ms. The top 5 precursor ions were fragmented in a data-dependent manner using higher-energy collisional dissociation (HCD) at 30% normalized collision energy and an MS2 resolution of 17,500. Data analysis was performed using Xcalibur 4.0 and Tracefinder 2.1 software (Thermo Fisher Scientific).

### Seahorse Assay

The day before the assay, the Seahorse cartridge was incubated overnight at 37° C. in XF calibrant. On the day of the assay, H9c2 cells were seeded at 15,000 cells per well, and cardiomyocytes at 10,000 cells per well, in a Seahorse 96-well plate. The plates were incubated at room temperature for 1 hour in glucose-free complete DMEM or RPMI without bicarbonate or phenol-red to ensure even cell distribution. Before analysis in the Seahorse XF96 Analyzer, the media volume in each well was adjusted to 175 μl. Sequential injections of 25 mM glucose, 2 μM oligomycin, 1.5 μM CCCP, and 50 mM 2DG, diluted in DMEM, were performed following the standard Seahorse protocol.

### α-KDGH Activity Measurement

α-KDGH activity was assessed using a colorimetric assay kit from Sigma (MAK-189) using 20-25 ug protein per sample in a microplate reader in kinetic mode for 10 min at 37° C. The reaction catalyzes the conversion of α-ketoglutarate to succinyl-CoA and NADH, therefore, a colorimetric product with absorbance at 450 nm is proportional to α-KDGH activity. One unit of α-KDGH activity is the amount of enzyme that generates 1.0 mol of NADH per min.

### α-KG Assay

The assay was performed using the Alpha-Ketoglutarate Assay Kit according to the manufacturer's instructions (Abcam, ab83431). All materials and reagents were equilibrated to room temperature prior to use. Samples and standards were prepared in duplicate, with sample volumes adjusted to 50 μL per well using α-KG Assay Buffer. A reaction mix was prepared and 50 μL was added to each standard and sample well. Background wells were prepared similarly, using a background reaction mix to account for any pyruvate interference. The plate was incubated at 37° C. for 30 minutes, protected from light. Following incubation, the optical density was measured at 570 nm for the colorimetric assay, or fluorescence was measured at Ex/Em=535/587 nm for the fluorometric assay. For data analysis, the mean absorbance value of the blanks was subtracted from all standard and sample readings to obtain corrected absorbance values. A standard curve was generated, and sample concentrations were extrapolated from this curve using the appropriate trendline equation. Samples producing signals higher than the highest standard were diluted and reassayed, with final concentrations adjusted by the corresponding dilution factor.

### Cytokine Measurement

Tissues from the RZ and BZ of the heart were compared using the Proteome Profiler Mouse Cytokine Array Kit, Panel A (Catalog #: ARY006, Biotechne). Reagents were brought to room temperature before use, and all procedures were conducted with appropriate precautions to avoid contamination. Membranes were prepared by placing them in a 4-Well Multi-dish with Array Buffer 6, followed by blocking and incubation with prepared samples mixed with the Mouse Cytokine Array Panel A Detection Antibody Cocktail. After an overnight incubation at 2-8° C., membranes were washed, treated with Streptavidin-HRP, and further incubated. After final washes, membranes were treated with Chemi Reagent Mix, carefully processed, and cytokine signals were detected using the iBright Imaging Systems (Thermo Fisher Scientific). Data analysis involved aligning the transparency overlay template with the array image, quantifying pixel densities using a scanner and image analysis software and comparing the average signal from duplicate spots on the arrays to assess relative changes in cytokine levels between RZ and BZ tissues.

### Coronary Ligation

The surgical protocol was performed as previously described (49). Briefly, mice were anesthetized with isoflurane with induction at 3% and maintenance at 1.5% to 2%. The animals were placed in a supine position and ECG leads were attached. The body temperature was monitored using a rectal probe and was maintained at 37° C. with heating pads throughout the experiment. A catheter was inserted into the trachea and was then attached to the mouse ventilator via a Y-shaped connector. The mice were ventilated at a tidal volume of 200 lL and a rate of 105 breaths/min using a rodent ventilator. The chest was then opened by an incision of the left fourth intercostal space. The left anterior descending artery was occluded with an 8-0 silk suture. Ischemia was confirmed by pallor of the anterior wall of the left ventricle and by ST-segment elevation and QRS widening on the ECG. After confirmation of ligation, the chest was closed in layers. The mice were kept warm with heating pads and on 100% oxygen via nasal cannula. Animals were given buprenorphine for postoperative pain. All animal procedures were followed in accordance with institutional guidelines.

### Injection of AA6

WT C57BL/6 and cs-TTP −/− mice were treated with (S)-2-[(2,6-dichlorobenzoyl)amino]succinic acid (AA6) according to the following schedule: mice received 50 mg/kg of AA6, dissolved in PBS (0.9% p/v NaCl), administered via intraperitoneal (i.p.) injections three times a week for 3 weeks. Following the treatment, the mice were subjected to either left anterior descending (LAD) coronary artery ligation or sham surgeries.

### ATP Measurement

Free ATP levels were measured using the ATP Bioluminescence Assay Kit CLS II (Roche Life Science Products). Samples were diluted with double-distilled water or buffer to achieve an ATP concentration within the optimal detection range of 10⁻⁷ to 10⁻¹⁰ M, with the pH adjusted to 7.6-8.0. ATP standards were prepared by serial dilution with double-distilled water in the same concentration range. ATP was extracted by adding 9 volumes of boiling 100 mM Tris, 4 mM EDTA (pH 7.75) to the cell suspension, incubating at 100° C. for 2 minutes, and then centrifuging at 1,000×g for 60 seconds. The supernatant was collected and kept on ice until measurement. For the assay, 50 μL of each sample or standard was transferred to a microplate well, and luciferase reagent was added by automated injection. After a 1-second delay, bioluminescence was measured with an integration time of 1 to 10 seconds. ATP concentrations were calculated by subtracting the blank (no ATP or no cells) and plotting the data on a log-log scale using the standard curve.

### Assessment of RNA-Seq Data

For the gene set enrichment analysis (GSEA), we used GSEA 4.3.3 software with the hallmark (H) gene set collection (50), encompassing 50 gene sets that represent well-defined biological states or processes. Genes differentially expressed in the border zone were separated into upregulated and downregulated categories (51, 52), with our analysis centered on the downregulated gene set. To improve the specificity of enrichment results, we restricted the gene set size to between 10 and 500 genes. The Benjamini-Hochberg correction was applied for multiple comparison adjustment to control the false discovery rate (53).

To investigate mRNA stability regulation in the BZ, we downloaded the REACTOME_REGULATION_OF_MRNA_STABILITY_BY_PROTEINS_THAT_BIND_AU_RICH_ELEMENTS gene set. Using RNA-seq data from the BZ (GSE183168), we examined upregulated genes within this specific gene set to identify proteins involved in mRNA binding and stability regulation. After filtering the list of genes, we applied a significance threshold with an adjusted p-value of 0.05 to isolate mRNA-binding proteins that were significantly upregulated in the BZ. (FIG. 3B).

### Statistical Analysis

Data are presented as mean±SD, unless otherwise stated. For a two-group comparison unpaired two-tailed Student's t-tests was used. For data with multiple groups (>2) or multiple treatments a one- or two-way ANOVA was used as indicated followed by Tukey's post-hoc test to determine p-values for individual comparisons. No statistical methods were used to predetermine sample size.

All statistical analysis was performed using Graphpad Prism 8.0. p<0.05 was considered statistically significant or ns=not significant. Survival experiment was performed in Prism 8 using comparison of survival curves with Log-rank (Mantel-Cox) test. For in vivo experiments, animals were assigned to experimental groups using simple randomization, without investigator blinding. Hierarchical clustering and heatmaps for metabolomics data were generated using MetaboAnalyst 4.0 statistical software (54).

### REFERENCES

1. Calcagno D M, Taghdiri N, Ninh V K, Mesfin J M, Toomu A, Sehgal R, et al. Single-cell and spatial transcriptomics of the infarcted heart define the dynamic onset of the border zone in response to mechanical destabilization. Nature Cardiovascular Research. 2022; 1(11):1039-55.
2. Arbustini E, Kramer C M, and Narula J. Arrhythmogenic Potential of Border Zone After Myocardial Infarction: Scar Is More Than Just a Healed Wound. JACC Cardiovasc Imaging. 2018; 11(4):573-6.
3. Calcagno D M, Taghdiri N, Ninh V K, Mesfin J M, Toomu A, Sehgal R, et al. Single-cell and spatial transcriptomics of the infarcted heart define the dynamic onset of the border zone in response to mechanical destabilization. Nat Cardiovasc Res. 2022; 1(11):1039-55.
4. Kuppe C, Ramirez Flores R O, Li Z, Hayat S, Levinson R T, Liao X, et al. Spatial multi-omic map of human myocardial infarction. Nature. 2022; 608(7924):766-77.
5. Talman V, and Ruskoaho H. Cardiac fibrosis in myocardial infarction—from repair and remodeling to regeneration. Cell Tissue Res. 2016; 365(3):563-81.
6. Jackson B M, Gorman J H, Moainie S L, Guy T S, Narula N, Narula J, et al. Extension of borderzone myocardium in postinfarction dilated cardiomyopathy. J Am Coll Cardiol. 2002; 40(6):1160-7; discussion 8-71.
7. Francis Stuart S D, De Jesus N M, Lindsey M L, and Ripplinger C M. The crossroads of inflammation, fibrosis, and arrhythmia following myocardial infarction. J Mol Cell Cardiol. 2016; 91:114-22.
8. Yang L, Gregorich Z R, Cai W, Zhang P, Young B, Gu Y, et al. Quantitative Proteomics and Immunohistochemistry Reveal Insights into Cellular and Molecular Processes in the Infarct Border Zone One Month after Myocardial Infarction. J Proteome Res. 2017; 16(5):2101-12.
9. Yin W, Chen Y, Wang W, Guo M, Tong L, Zhang M, et al. Macrophage-mediated heart repair and remodeling: A promising therapeutic target for post-myocardial infarction heart failure. J Cell Physiol. 2024:e31372.
10. Frangogiannis N G. The reparative function of cardiomyocytes in the infarcted myocardium. Cell Metab. 2015; 21(6):797-8.
11. Dong S, Cheng Y, Yang J, Li J, Liu X, Wang X, et al. MicroRNA expression signature and the role of microRNA-21 in the early phase of acute myocardial infarction. J Biol Chem. 2009; 284(43):29514-25.
12. Glass C, and Singla D K. MicroRNA-1 transfected embryonic stem cells enhance cardiac myocyte differentiation and inhibit apoptosis by modulating the PTEN/Akt pathway in the infarcted heart. Am J Physiol Heart Circ Physiol. 2011; 301(5):H2038-49.
13. Lodrini A M, and Goumans M J. Cardiomyocytes Cellular Phenotypes After Myocardial Infarction. Front Cardiovasc Med. 2021; 8:750510.
14. Peng L, Fu C, Liang Z, Zhang Q, Xiong F, Chen L, et al. Pulsed Electromagnetic Fields Increase Angiogenesis and Improve Cardiac Function After Myocardial Ischemia in Mice. Circ J 2020; 84(2):186-93.
15. Chouchani E T, Pell V R, Gaude E, Aksentijevic D, Sundier S Y, Robb E L, et al. Ischaemic accumulation of succinate controls reperfusion injury through mitochondrial ROS. Nature. 2014; 515(7527):431-5.
16. Zhang J, Wang Y T, Miller J H, Day M M, Munger J C, and Brookes P S. Accumulation of Succinate in Cardiac Ischemia Primarily Occurs via Canonical Krebs Cycle Activity. Cell Rep. 2018; 23(9):2617-28.
17. Li X, Wu F, Gunther S, Looso M, Kuenne C, Zhang T, et al. Inhibition of fatty acid oxidation enables heart regeneration in adult mice. Nature. 2023; 622(7983):619-26.
18. Brooks S A, and Blackshear P J. Tristetraprolin (TTP): interactions with mRNA and proteins, and current thoughts on mechanisms of action. Biochimica et biophysica acta. 2013; 1829(6-7):666-79.
19. Lai W S, Stumpo D J, and Blackshear P J. Rapid insulin-stimulated accumulation of an mRNA encoding a proline-rich protein. J Biol Chem. 1990; 265(27):16556-63.
20. Taylor G A, Carballo E, Lee D M, Lai W S, Thompson M J, Patel D D, et al. A pathogenetic role for TNF alpha in the syndrome of cachexia, arthritis, and autoimmunity resulting from tristetraprolin (TTP) deficiency. Immunity. 1996; 4(5):445-54.
21. Carballo E, Gilkeson G S, and Blackshear P J. Bone marrow transplantation reproduces the tristetraprolin-deficiency syndrome in recombination activating gene-2 (−/−) mice. Evidence that monocyte/macrophage progenitors may be responsible for TNFalpha overproduction. The Journal of clinical investigation. 1997; 100(5):986-95.
22. Carballo E, Lai W S, and Blackshear P J. Feedback inhibition of macrophage tumor necrosis factor-alpha production by tristetraprolin. Science. 1998; 281(5379):1001-5.
23. Bayeva M, Khechaduri A, Puig S, Chang H C, Patial S, Blackshear P J, et al. mTOR regulates cellular iron homeostasis through tristetraprolin. Cell Metab. 2012; 16(5):645-57.
24. Bouchard L, Tchernof A, Deshaies Y, Marceau S, Lescelleur O, Biron S, et al. ZFP36: a promising candidate gene for obesity-related metabolic complications identified by converging genomics. Obesity surgery. 2007; 17(3):372-82.
25. Bouchard L, Vohl M C, Deshaies Y, Rheaume C, Daris M, and Tchernof A. Visceral adipose tissue zinc finger protein 36 mRNA levels are correlated with insulin, insulin resistance index, and adiponectinemia in women. European journal of endocrinology. 2007; 157(4):451-7.
26. Puig S, Vergara S V, and Thiele D J. Cooperation of two mRNA-binding proteins drives metabolic adaptation to iron deficiency. Cell Metab. 2008; 7(6):555-64.
27. Sato T, Chang H C, Bayeva M, Shapiro J S, Ramos-Alonso L, Kouzu H, et al. mRNA-binding protein tristetraprolin is essential for cardiac response to iron deficiency by regulating mitochondrial function. Proceedings of the National Academy of Sciences of the United States of America. 2018; 115(27):E6291-E300.
28. Nian M, Lee P, Khaper N, and Liu P. Inflammatory Cytokines and Postmyocardial Infarction Remodeling. Circulation Research. 2004; 94(12):1543-53.
29. Matter M A, Paneni F, Libby P, Frantz S, Stahli B E, Templin C, et al. Inflammation in acute myocardial infarction: the good, the bad and the ugly. European Heart Journal. 2024; 45(2):89-103.
30. Huang S, and Frangogiannis N G. Anti-inflammatory therapies in myocardial infarction: failures, hopes and challenges. British Journal of Pharmacology. 2018; 175(9):1377-400.
31. Dimosiari A, Patoulias D, Kitas G D, and Dimitroulas T. Do Interleukin-1 and Interleukin-6 Antagonists Hold Any Place in the Treatment of Atherosclerotic Cardiovascular Disease and Related Co-Morbidities? An Overview of Available Clinical Evidence. Journal of clinical medicine. 2023; 12(4).
32. King K R, Aguirre A D, Ye Y-X, Sun Y, Roh J D, Ng R P, et al. IRF3 and type I interferons fuel a fatal response to myocardial infarction. Nature Medicine. 2017; 23(12):1481-7.
33. Saxena A, Russo I, and Frangogiannis N G. Inflammation as a therapeutic target in myocardial infarction: learning from past failures to meet future challenges. Translational Research. 2016; 167(1):152-66.
34. Huse C, Anstensrud A K, Michelsen A E, Ueland T, Broch K, Woxholt S, et al. Interleukin-6 inhibition in ST-elevation myocardial infarction: Immune cell profile in the randomised ASSAIL-MI trial. EBioMedicine. 2022; 80:104013.
35. Chouchani E T, Pell V R, Gaude E, Aksentijevid D, Sundier S Y, Robb E L, et al. Ischaemic accumulation of succinate controls reperfusion injury through mitochondrial ROS. Nature. 2014; 515(7527):431-5.
36. van Duijvenboden K, de Bakker D E M, Man J C K, Janssen R, Gunthel M, Hill M C, et al. Conserved NPPB+ Border Zone Switches From MEF2- to AP-1-Driven Gene Program. Circulation. 2019; 140(10):864-79.
37. Prasad K M, Smith R S, Xu Y, and French B A. A single direct injection into the left ventricular wall of an adeno-associated virus 9 (AAV9) vector expressing extracellular superoxide dismutase from the cardiac troponin-T promoter protects mice against myocardial infarction. The journal of gene medicine. 2011; 13(6):333-41.
38. Fenwick P S, Macedo P, Kilty I C, Barnes P J, and Donnelly L E. Effect of JAK Inhibitors on Release of CXCL9, CXCL10 and CXCL11 from Human Airway Epithelial Cells. PLoS One. 2015; 10(6):e0128757.
39. Li X, Wu F, Gunther S, Looso M, Kuenne C, Zhang T, et al. Publisher Correction: Inhibition of fatty acid oxidation enables heart regeneration in adult mice. Nature. 2023.
40. Casanova J-L, MacMicking JD, and Nathan C F. Interferon-7 and infectious diseases: Lessons and prospects. Science. 2024; 384(6693):eadl2016.
41. Hoyer F F, and Nahrendorf M. Oxford University Press; 2019:1815-6.
42. King K R, Aguirre A D, Ye Y X, Sun Y, Roh J D, Ng R P, Jr., et al. IRF3 and type I interferons fuel a fatal response to myocardial infarction. Nature medicine. 2017; 23(12):1481-7.
43. Calcagno D M, Ng R P, Toomu A, Zhang C, Huang K, Aguirre A D, et al. The myeloid type I interferon response to myocardial infarction begins in bone marrow and is regulated by Nrf2-activated macrophages. Science Immunology. 2020; 5(51):eaaz1974.
44. Cao D J, Schiattarella G G, Villalobos E, Jiang N, May H I, Li T, et al. Cytosolic DNA Sensing Promotes Macrophage Transformation and Governs Myocardial Ischemic Injury. Circulation. 2018; 137(24):2613-34.
45. Ngwenyama N, Kaur K, Bugg D, Theall B, Aronovitz M, Berland R, et al. Antigen presentation by cardiac fibroblasts promotes cardiac dysfunction. Nat Cardiovasc Res. 2022; 1(8):761-74.
46. Günthel M, van Duijvenboden K, de Bakker D E M, Hooijkaas I B, Bakkers J, Barnett P, et al. Epigenetic State Changes Underlie Metabolic Switch in Mouse Post-Infarction Border Zone Cardiomyocytes. Journal of cardiovascular development and disease. 2021; 8(11).
47. Fu Y, Jiang W, Zhao Y, Huang Y, Zhang H, Wang H, et al. A Simple and Efficient Method for In Vivo Cardiac-specific Gene Manipulation by Intramyocardial Injection in Mice. J Vis Exp. 2018(134).
48. Sawicki K T, Chang H C, Shapiro J S, Bayeva M, De Jesus A, Finck B N, et al. Hepatic tristetraprolin promotes insulin resistance through RNA destabilization of FGF21. JCI Insight. 2018; 3(13).
49. Sawicki K T, Shang M, Wu R, Chang H C, Khechaduri A, Sato T, et al. Increased Heme Levels in the Heart Lead to Exacerbated Ischemic Injury. J Am Heart Assoc. 2015; 4(8):e002272.
50. Liberzon A, Birger C, Thorvaldsdóttir H, Ghandi M, Mesirov J P, and Tamayo P. The Molecular Signatures Database (MSigDB) hallmark gene set collection. Cell systems. 2015; 1(6):417-25.W
51. Hong G, Zhang W, Li H, Shen X, and Guo Z. Separate enrichment analysis of pathways for up- and downregulated genes. Journal of the Royal Society Interface. 2014; 11(92):20130950.
52. Gao W, Kim M W, Dykstra T, Du S, Boskovic P, Lichti C F, et al. Engineered T cell therapy for central nervous system injury. Nature. 2024; 634(8034):693-701.
53. Yu G, Wang L-G, Han Y, and He Q-Y. clusterProfiler: an R package for comparing biological themes among gene clusters. Omics: a journal of integrative biology. 2012; 16(5):284-7.
54. Chong J, Wishart D S, and Xia J. Using MetaboAnalyst 4.0 for Comprehensive and Integrative Metabolomics Data Analysis. Curr Protoc Bioinformatics. 2019; 68(1):e86.

What is claimed:

1. A method of treating myocardial infarction (MI) in a subject in need, the method comprising administering to the subject a therapeutically effective amount of at least one of an interferon-gamma (IFNγ) inhibitor and an interleukin 6 (IL-6) inhibitor.

2. The method of claim 1, wherein the method comprises administering the IFNγ inhibitor.

3. The method of claim 1, wherein the IFNγ inhibitor reduces IFNγ expression.

4. The method of claim 1, wherein the IFNγ inhibitor reduces IFNγ activity.

5. The method of claim 1, wherein the IFNγ inhibitor comprises at least one of an antibody, a small molecule drug, a peptide, a gene silencing agent, a protein degrader, and a cytokine.

6. The method of claim 5, wherein the IFNγ inhibitor comprises an IFNγ antibody.

7. The method of claim 6, wherein the IFNγ antibody is Emapalumab.

8. The method of claim 6, wherein between about 25 ng/kg and about 50 ng/kg of the IFNγ antibody is administered.

9. The method of claim 8, wherein about 50 ng/kg of the IFNγ antibody is administered.

10. The method of claim 6, wherein the IFNγ antibody is administered subcutaneously or intravenously.

11. The method of claim 5, wherein the IFNγ inhibitor comprises a Jak/Stat inhibitor.

12. The method of claim 11, wherein the Jak/Stat inhibitor comprises Ruxolitinib.

13. The method of claim 1, wherein the IFNγ inhibitor is administered at least once.

14. The method of claim 1, wherein the inhibitor is administered between about 1 day and about 3 days following an MI.

15. The method of claim 1, further comprising administering at least one additional therapy for myocardial infarction.

16. The method of claim 15, wherein the at least one additional therapy is selected from the group consisting of an anticoagulant, an antianginal, a narcotic, a beta blocker, a statin, an ACE inhibitor, a surgery, a stent, and an angioplasty.

17. The method of claim 1, wherein the subject is a human.

18. The method of claim 1, wherein the method comprises administering the IL-6 inhibitor.

19. The method of claim 1, wherein the subject expresses elevated TTP levels in border zone cardiomyocytes.

20. The method of claim 1, wherein the subject express reduced α-KGDH activity.
